SEQUENCE LISTING (55 sequences)

SEQ ID NO: 1 - protein, 227 aa, Artificial Sequence
  GlxR_amino acid
MEGVQEILSRAGIFQGVDPTAVNNLIQDMETVRFPRGATIFDEGEPGDRLYIITSGKVKLARHAPDGRENLLTIMGPSDMFGELSIFDPGPRTSSAVCVTEVHAATMNSDMLRNWVADHPAIAEQLLRVLARRLRRTNASLADLIFTDVPGRVAKTLLQLANRFGTQEAGALRVNHDLTQEEIAQLVGASRETVNKALATFAHRGWIRLEGKSVLIVDTEHLARRAR

SEQ ID NO: 2 - DNA, 684 bp, Artificial Sequence
  glxR_nucleic acid
gtggaaggtgtacaggagatcctgtcgcgcgccggaatttttcaaggcgttgacccaacggcagtcaataacctcatccaggatatggagaccgttcgcttcccacgcggagcaaccatcttcgacgagggcgagccaggtgaccgcctttacatcatcacctccggcaaagtgaagcttgcgcgccacgcaccggacggccgcgaaaacctgctgaccatcatgggtccttccgacatgttcggtgagctctccatcttcgacccaggcccacgcacctcctctgcagtgtgtgtcaccgaagttcatgcagcaaccatgaactctgacatgctgcgcaactgggtagctgaccacccagctatcgctgagcagctcctgcgcgttctggctcgtcgtctgcgtcgcaccaacgcttccctggctgacctcatcttcaccgacgtcccaggccgcgttgctaagacccttctgcagctggctaaccgcttcggcacccaagaagctggcgcgctgcgcgtgaaccacgacctcactcaggaagaaatcgcacagctcgtcggtgcttcccgtgaaactgtgaataaggctcttgcaacgttcgcacaccgtggctggattcgcctcgagggcaagtccgtcctcattgtggacaccgagcatttggcacgtcgcgctcgataa

SEQ ID NO: 3 - protein, 227 aa, Artificial Sequence
  GlxR_E45A
MEGVQEILSRAGIFQGVDPTAVNNLIQDMETVRFPRGATIFDEGAPGDRLYIITSGKVKLARHAPDGRENLLTIMGPSDMFGELSIFDPGPRTSSAVCVTEVHAATMNSDMLRNWVADHPAIAEQLLRVLARRLRRTNASLADLIFTDVPGRVAKTLLQLANRFGTQEAGALRVNHDLTQEEIAQLVGASRETVNKALATFAHRGWIRLEGKSVLIVDTEHLARRAR

SEQ ID NO: 4 - protein, 227 aa, Artificial Sequence
  GlxR_E45S
MEGVQEILSRAGIFQGVDPTAVNNLIQDMETVRFPRGATIFDEGSPGDRLYIITSGKVKLARHAPDGRENLLTIMGPSDMFGELSIFDPGPRTSSAVCVTEVHAATMNSDMLRNWVADHPAIAEQLLRVLARRLRRTNASLADLIFTDVPGRVAKTLLQLANRFGTQEAGALRVNHDLTQEEIAQLVGASRETVNKALATFAHRGWIRLEGKSVLIVDTEHLARRAR

SEQ ID NO: 5 - protein, 227 aa, Artificial Sequence
  GlxR_E45F
MEGVQEILSRAGIFQGVDPTAVNNLIQDMETVRFPRGATIFDEGFPGDRLYIITSGKVKLARHAPDGRENLLTIMGPSDMFGELSIFDPGPRTSSAVCVTEVHAATMNSDMLRNWVADHPAIAEQLLRVLARRLRRTNASLADLIFTDVPGRVAKTLLQLANRFGTQEAGALRVNHDLTQEEIAQLVGASRETVNKALATFAHRGWIRLEGKSVLIVDTEHLARRAR

SEQ ID NO: 6 - protein, 227 aa, Artificial Sequence
  GlxR_E45K
MEGVQEILSRAGIFQGVDPTAVNNLIQDMETVRFPRGATIFDEGKPGDRLYIITSGKVKLARHAPDGRENLLTIMGPSDMFGELSIFDPGPRTSSAVCVTEVHAATMNSDMLRNWVADHPAIAEQLLRVLARRLRRTNASLADLIFTDVPGRVAKTLLQLANRFGTQEAGALRVNHDLTQEEIAQLVGASRETVNKALATFAHRGWIRLEGKSVLIVDTEHLARRAR

SEQ ID NO: 7 - protein, 227 aa, Artificial Sequence
  GlxR_D66A
MEGVQEILSRAGIFQGVDPTAVNNLIQDMETVRFPRGATIFDEGEPGDRLYIITSGKVKLARHAPAGRENLLTIMGPSDMFGELSIFDPGPRTSSAVCVTEVHAATMNSDMLRNWVADHPAIAEQLLRVLARRLRRTNASLADLIFTDVPGRVAKTLLQLANRFGTQEAGALRVNHDLTQEEIAQLVGASRETVNKALATFAHRGWIRLEGKSVLIVDTEHLARRAR

SEQ ID NO: 8 - protein, 227 aa, Artificial Sequence
  GlxR_D66F
MEGVQEILSRAGIFQGVDPTAVNNLIQDMETVRFPRGATIFDEGEPGDRLYIITSGKVKLARHAPFGRENLLTIMGPSDMFGELSIFDPGPRTSSAVCVTEVHAATMNSDMLRNWVADHPAIAEQLLRVLARRLRRTNASLADLIFTDVPGRVAKTLLQLANRFGTQEAGALRVNHDLTQEEIAQLVGASRETVNKALATFAHRGWIRLEGKSVLIVDTEHLARRAR

SEQ ID NO: 9 - protein, 227 aa, Artificial Sequence
  GlxR_D66K
MEGVQEILSRAGIFQGVDPTAVNNLIQDMETVRFPRGATIFDEGEPGDRLYIITSGKVKLARHAPKGRENLLTIMGPSDMFGELSIFDPGPRTSSAVCVTEVHAATMNSDMLRNWVADHPAIAEQLLRVLARRLRRTNASLADLIFTDVPGRVAKTLLQLANRFGTQEAGALRVNHDLTQEEIAQLVGASRETVNKALATFAHRGWIRLEGKSVLIVDTEHLARRAR

SEQ ID NO: 10 - protein, 227 aa, Artificial Sequence
  GlxR_T166D
MEGVQEILSRAGIFQGVDPTAVNNLIQDMETVRFPRGATIFDEGEPGDRLYIITSGKVKLARHAPDGRENLLTIMGPSDMFGELSIFDPGPRTSSAVCVTEVHAATMNSDMLRNWVADHPAIAEQLLRVLARRLRRTNASLADLIFTDVPGRVAKTLLQLANRFGDQEAGALRVNHDLTQEEIAQLVGASRETVNKALATFAHRGWIRLEGKSVLIVDTEHLARRAR

SEQ ID NO: 11 - protein, 227 aa, Artificial Sequence
  GlxR_T166F
MEGVQEILSRAGIFQGVDPTAVNNLIQDMETVRFPRGATIFDEGEPGDRLYIITSGKVKLARHAPDGRENLLTIMGPSDMFGELSIFDPGPRTSSAVCVTEVHAATMNSDMLRNWVADHPAIAEQLLRVLARRLRRTNASLADLIFTDVPGRVAKTLLQLANRFGFQEAGALRVNHDLTQEEIAQLVGASRETVNKALATFAHRGWIRLEGKSVLIVDTEHLARRAR

SEQ ID NO: 12 - protein, 227 aa, Artificial Sequence
  GlxR_T166K
MEGVQEILSRAGIFQGVDPTAVNNLIQDMETVRFPRGATIFDEGEPGDRLYIITSGKVKLARHAPDGRENLLTIMGPSDMFGELSIFDPGPRTSSAVCVTEVHAATMNSDMLRNWVADHPAIAEQLLRVLARRLRRTNASLADLIFTDVPGRVAKTLLQLANRFGKQEAGALRVNHDLTQEEIAQLVGASRETVNKALATFAHRGWIRLEGKSVLIVDTEHLARRAR

SEQ ID NO: 13 - protein, 227 aa, Artificial Sequence
  GlxR_E168A
MEGVQEILSRAGIFQGVDPTAVNNLIQDMETVRFPRGATIFDEGEPGDRLYIITSGKVKLARHAPDGRENLLTIMGPSDMFGELSIFDPGPRTSSAVCVTEVHAATMNSDMLRNWVADHPAIAEQLLRVLARRLRRTNASLADLIFTDVPGRVAKTLLQLANRFGTQAAGALRVNHDLTQEEIAQLVGASRETVNKALATFAHRGWIRLEGKSVLIVDTEHLARRAR

SEQ ID NO: 14 - protein, 227 aa, Artificial Sequence
  GlxR_E168F
MEGVQEILSRAGIFQGVDPTAVNNLIQDMETVRFPRGATIFDEGEPGDRLYIITSGKVKLARHAPDGRENLLTIMGPSDMFGELSIFDPGPRTSSAVCVTEVHAATMNSDMLRNWVADHPAIAEQLLRVLARRLRRTNASLADLIFTDVPGRVAKTLLQLANRFGTQFAGALRVNHDLTQEEIAQLVGASRETVNKALATFAHRGWIRLEGKSVLIVDTEHLARRAR

SEQ ID NO: 15 - protein, 227 aa, Artificial Sequence
  GlxR_E168K
MEGVQEILSRAGIFQGVDPTAVNNLIQDMETVRFPRGATIFDEGEPGDRLYIITSGKVKLARHAPDGRENLLTIMGPSDMFGELSIFDPGPRTSSAVCVTEVHAATMNSDMLRNWVADHPAIAEQLLRVLARRLRRTNASLADLIFTDVPGRVAKTLLQLANRFGTQKAGALRVNHDLTQEEIAQLVGASRETVNKALATFAHRGWIRLEGKSVLIVDTEHLARRAR

SEQ ID NO: 16 - DNA, 35 bp, Artificial Sequence
  glxR L1
gaattcgagctcggtaccctgagaccccacgcgag

SEQ ID NO: 17 - DNA, 36 bp, Artificial Sequence
  glxR R2
gactctagaggatccccatcgacggtgtcaccccac

SEQ ID NO: 18 - DNA, 38 bp, Artificial Sequence
  glxR(E45A) L2
gtaaaggcggtcacctggcgcgccctcgtcgaagatgg

SEQ ID NO: 19 - DNA, 38 bp, Artificial Sequence
  glxR(E45A) R1
ccatcttcgacgagggcgcgccaggtgaccgcctttac

SEQ ID NO: 20 - DNA, 21 bp, Artificial Sequence
  pDZ_F (pDZ vector sequencing primer_forward)
tattacgccagctggcgaaag

SEQ ID NO: 21 - DNA, 21 bp, Artificial Sequence
  pDZ_R (pDZ vector sequencing primer_reverse)
ttccggctcgtatgttgtgtg

SEQ ID NO: 22 - DNA, 38 bp, Artificial Sequence
  glxR(E45S) L2
gtaaaggcggtcacctggagagccctcgtcgaagatgg

SEQ ID NO: 23 - DNA, 38 bp, Artificial Sequence
  glxR(E45S) R1
ccatcttcgacgagggctctccaggtgaccgcctttac

SEQ ID NO: 24 - DNA, 38 bp, Artificial Sequence
  glxR(E45F) L2
gtaaaggcggtcacctggaaagccctcgtcgaagatgg

SEQ ID NO: 25 - DNA, 38 bp, Artificial Sequence
  glxR(E45F) R1
ccatcttcgacgagggctttccaggtgaccgcctttac

SEQ ID NO: 26 - DNA, 38 bp, Artificial Sequence
  glxR(E45K) L2
gtaaaggcggtcacctggcttgccctcgtcgaagatgg

SEQ ID NO: 27 - DNA, 38 bp, Artificial Sequence
  glxR(E45K) R1
ccatcttcgacgagggcaagccaggtgaccgcctttac

SEQ ID NO: 28 - DNA, 34 bp, Artificial Sequence
  glxR(D66A) L2
ggttttcgcggcctgccggtgcgtggcgcgcaag

SEQ ID NO: 29 - DNA, 34 bp, Artificial Sequence
  glxR(D66A) R1
gcgccacgcaccggcaggccgcgaaaacctgctg

SEQ ID NO: 30 - DNA, 34 bp, Artificial Sequence
  glxR(D66F) L2
cagcaggttttcgcggccaaacggtgcgtggcgc

SEQ ID NO: 31 - DNA, 34 bp, Artificial Sequence
  glxR(D66F) R1
gcgccacgcaccgtttggccgcgaaaacctgctg

SEQ ID NO: 32 - DNA, 35 bp, Artificial Sequence
  glxR(D66K) L2
ggttttcgcggcctttccggtgcgtggcgcgcaag

SEQ ID NO: 33 - DNA, 34 bp, Artificial Sequence
  glxR(D66K) R1
gcgccacgcaccgaaaggccgcgaaaacctgctg

SEQ ID NO: 34 - DNA, 34 bp, Artificial Sequence
  glxR(T166D) L2
gccagcttcttggtcgccgaagcggttagccagc

SEQ ID NO: 35 - DNA, 33 bp, Artificial Sequence
  glxR(T166D) R1
aaccgcttcggcgaccaagaagctggcgcgctg

SEQ ID NO: 36 - DNA, 34 bp, Artificial Sequence
  glxR(T166F) L2
gccagcttcttggaagccgaagcggttagccagc

SEQ ID NO: 37 - DNA, 33 bp, Artificial Sequence
  glxR(T166F) R1
aaccgcttcggcttccaagaagctggcgcgctg

SEQ ID NO: 38 - DNA, 34 bp, Artificial Sequence
  glxR(T166K) L2
gccagcttcttgtttgccgaagcggttagccagc

SEQ ID NO: 39 - DNA, 33 bp, Artificial Sequence
  glxR(T166K) R1
aaccgcttcggcaaacaagaagctggcgcgctg

SEQ ID NO: 40 - DNA, 34 bp, Artificial Sequence
  glxR(E168A) L2
agcgcgccagcagcttgggcgccgaagcggttag

SEQ ID NO: 41 - DNA, 34 bp, Artificial Sequence
  glxR(E168A) R1
cttcggcgcccaagctgctggcgcgctgcgcgtg

SEQ ID NO: 42 - DNA, 34 bp, Artificial Sequence
  glxR(E168F) L2
agcgcgccagcaaattgggcgccgaagcggttag

SEQ ID NO: 43 - DNA, 34 bp, Artificial Sequence
  glxR(E168F) R1
cttcggcgcccaatttgctggcgcgctgcgcgtg

SEQ ID NO: 44 - DNA, 34 bp, Artificial Sequence
  glxR(E168K) L2
agcgcgccagccttttgggcgccgaagcggttag

SEQ ID NO: 45 - DNA, 34 bp, Artificial Sequence
  glxR(E168K) R1
cttcggcgcccaaaaggctggcgcgctgcgcgtg

SEQ ID NO: 46 - protein, 421 aa, Artificial Sequence
  LysC_L377K
MALVVQKYGGSSLESAERIRNVAERIVATKKAGNDVVVVCSAMGDTTDELLELAAAVNPVPPAREMDMLLTAGERISNALVAMAIESLGAEAQSFTGSQAGVLTTERHGNARIVDVTPGRVREALDEGKICIVAGFQGVNKETRDVTTLGRGGSDTTAVALAAALNADVCEIYSDVDGVYTADPRIVPNAQKLEKLSFEEMLELAAVGSKILVLRSVEYARAFNVPLRVRSSYSNDPGTLIAGSMEDIPVEEAVLTGVATDKSEAKVTVLGISDKPGEAAKVFRALADAEINIDMVLQNVSSVEDGTTDITFTCPRSDGRRAMEILKKLQVQGNWTNVLYDDQVGKVSLVGAGMKSHPGVTAEFMEALRDVNVNIEKISTSEIRISVLIREDDLDAAARALHEQFQLGGEDEAVVYAGTGR

SEQ ID NO: 47 - DNA, 34 bp, Artificial Sequence
  lysC L1
tcgagctcggtacccgctgcgcagtgttgaatac

SEQ ID NO: 48 - DNA, 34 bp, Artificial Sequence
  lysC R2
ctctagaggatccccgttcacctcagagacgatt

SEQ ID NO: 49 - DNA, 30 bp, Artificial Sequence
  lysC(L377K) L2
tggaaatcttttcgatgttcacgttgacat

SEQ ID NO: 50 - DNA, 30 bp, Artificial Sequence
  lysC(L377K) R1
atgtcaacgtgaacatcgaaaagatttcca

SEQ ID NO: 51 - protein, 445 aa, Artificial Sequence
  Hom_R407H
MTSASAPSFNPGKGPGSAVGIALLGFGTVGTEVMRLMTEYGDELAHRIGGPLEVRGIAVSDISKPREGVAPELLTEDAFALIEREDVDIVVEVIGGIEYPREVVLAALKAGKSVVTANKALVAAHSAELADAAEAANVDLYFEAAVAGAIPVVGPLRRSLAGDQIQSVMGIVNGTTNFILDAMDSTGADYADSLAEATRLGYAEADPTADVEGHDAASKAAILASIAFHTRVTADDVYCEGISNISAADIEAAQQAGHTIKLLAICEKFTNKEGKSAISARVHPTLLPVSHPLASVNKSFNAIFVEAEAAGRLMFYGNGAGGAPTASAVLGDVVGAARNKVHGGRAPGESTYANLPIADFGETTTRYHLDMDVEDRVGVLAELASLFSEQGISLRTIRQEERDDDAHLIVVTHSALESDLSRTVELLKAKPVVKAINSVIRLERD

SEQ ID NO: 52 - DNA, 34 bp, Artificial Sequence
  hom L1
atcctctagagtcgacccaactgcagacgtcgaa

SEQ ID NO: 53 - DNA, 35 bp, Artificial Sequence
  hom R2
atgcctgcaggtcgacatagacagatttgtccacg

SEQ ID NO: 54 - DNA, 35 bp, Artificial Sequence
  hom(R407H) L2
gtgaccacgatcagatgtgcatcatcatcgcgctc

SEQ ID NO: 55 - DNA, 35 bp, Artificial Sequence
  hom(R407H) R1
gcgatgatgatgcacatctgatcgtggtcacccac

(19) United States
(12) Patent Application Publication
(10) Pub. No.: US 2025/0326803 A1
(43) Pub. Date: Oct. 23, 2025

(54) GLXR PROTEIN VARIANT OR THREONINE PRODUCTION METHOD USING SAME

(71) Applicant: CJ CheilJedang Corporation, Seoul (KR)

(72) Inventors: Yee-Seul SO, Seoul (KR); Su Yon KWON, Seoul (KR); Kwang Woo LEE, Seoul (KR); Jihyun SHIM, Seoul (KR)

(73) Assignee: CJ CheilJedang Corporation, Seoul (KR)

(21) Appl. No.: 18/271,292

(22) PCT Filed: Jan. 6, 2022
(86) PCT No.: PCT/KR2022/000204

§ 371 Date: Jul. 7, 2023

(30) Foreign Application Priority Data
Jan. 11, 2021 (KR) 10-2021-0003609

(51) Int. Cl. C07K 14/34; C12N 1/20; C12N 15/77; C12P 13/08

(52) U.S. Cl. CPC C07K 14/34; C12N 1/20; C12N 15/77; C12P 13/08

(57) ABSTRACT

The present application relates to a GlxR protein variant or a threonine production method using same, and the GlxR protein variant according to one embodiment is introduced into a microorganism to reduce by-product output and remarkably increase threonine production capacity.

### TECHNICAL FIELD

### Cross-Reference to Related Application(S)

The present application claims the benefit of the priority based on Korean Patent Application No. 10-2021-0003609 filed on Jan. 11, 2021, and the entire contents of the document of the corresponding Korean patent application are incorporated as a part of the present description.

The present application relates to a GlxR protein variant or a threonine production method using the same.

### Background Art

L-threonine is an essential amino acid, and is used as a feed or food additive, a synthetic raw material for pharmaceuticals and an infusion solution for medicine. L-threonine is mainly produced by microbial fermentation technology, and for example, it may be produced using an Escherichia, Corynebacterium, Serratia, or Providencia sp. microorganism.

Recently, many attempts have been made to improve a preparation method of L-threonine using a Corynebacterium sp. strain. As gene recombination technology develops, technologies for development of more improved L-threonine producing strains have been reported by introducing site-specific gene substitution, gene amplification and deletion, and the like for a L-threonine producing strain developed by random mutagenesis. In addition, there is also an attempt to introduce a foreign gene from other bacteria.

Despite these efforts, development of a technology for improving the productivity of useful substances such as L-threonine is still required.

### PRIOR ART

### Patent Document

(Patent document 1) U.S. Patent Publication No. 2016-0369310

### DISCLOSURE

### Technical Problem

One example provides a novel GlxR protein variant. The GlxR protein variant may be a polypeptide in which an amino acid at a position of one or more selected from the group consisting of the 45^{th}, 66^{th}, 166^{th} and 168^{th} from the N-terminus in the amino acid sequence of SEQ ID NO: 1 and/or corresponding thereto (at one or more positions selected above) is substituted with other amino acid. The polypeptide may have the function of a major transcriptional regulator regulating a metabolic process.

Another example provides a polynucleotide encoding the GlxR protein variant.

Other example provides a vector comprising the polynucleotide.

Other example provides a microorganism comprising the GlxR protein variant, the polynucleotide, a vector comprising the polynucleotide, or a combination thereof.

Other example provides a production method of threonine comprising culturing the microorganism in a medium.

The microorganism may be a Corynebacterium sp. microorganism.

### Technical Solution

One example provided in the present application provides a technology related to developing a strain with improved (increased) production of threonine by improving GlxR protein and/org GlxR gene encoding this. In one example, the threonine may be L-threonine.

One aspect of the present application to achieve the purpose may provide a GlxR protein variant, in which an amino acid residue at a position of one or more selected from the group consisting of the 45^{th}, 66^{th}, 166^{th} and 168^{th} from the N-terminus in the amino acid sequence of GlxR protein and/or corresponding position thereto is substituted with other kind of amino acid residue.

Herein, “GlxR (CRP-like cyclic AMP-dependent global transcriptional regulator, for example, Cg0350)” may mean a global regulator which involves in not only carbon metabolism but also various cell functions to regulate various genes, and for example, it may regulate expression of a gene encoding glyoxylic acid bypass enzymes, isocitrate lyase and/or malate synthase (for example, aceA (cg2560) and aceB (cg2559)).

The GlxR protein may comprise the amino acid sequence of SEQ ID NO: 1 or consist of the amino acid sequence of SEQ ID NO: 1, and the gIxR gene encoding the GlxR protein may comprise the nucleic acid sequence of SEQ ID NO: 2 or consist of the nucleic acid sequence of SEQ ID NO: 2. The amino acid sequence of SEQ ID NO: 1 and the nucleic acid sequence of SEQ ID NO: 2 were described in Table 1 below.

The amino acid sequence of the GlxR protein and the nucleotide sequence (nucleic acid sequence) of the gene encoding the GlxR protein (for example, gIxR) were easily obtained from database known in the art such as U.S. National Center for Biotechnology Information (NCBI) and Japanese DNA databank (DDBJ), and for example, it may be GenBank Accession No. WP_003855810.1.

TABLE 1

| Name | Sequence | SEQ ID NO |
|---|---|---|
| GlxR_amino | MEGVQEILSRAGIFQGVDPTAVNNLIQDMETVRFPRGA | SEQ ID NO: 1 |
| acid | TIFDEGEPGDRLYIITSGKVKLARHAPDGRENLLTIMGP | |
| | SDMFGELSIFDPGPRTSSAVCVTEVHAATMNSDMLRN | |
| | WVADHPAIAEQLLRVLARRLRRTNASLADLIFTDVPGR | |
| | VAKTLLQLANRFGTQEAGALRVNHDLTQEEIAQLVGAS | |
| | RETVNKALATFAHRGWIRLEGKSVLIVDTEHLARRAR | |
| glxR_nucleic | GTGGAAGGTGTACAGGAGATCCTGTCGCGCGCCGG | SEQ ID NO: 2 |
| acid | AATTTTTCAAGGCGTTGACCCAACGGCAGTCAATAA | |
| | CCTCATCCAGGATATGGAGACCGTTCGCTTCCCACG | |
| | CGGAGCAACCATCTTCGACGAGGGCGAGCCAGGTG | |
| | ACCGCCTTTACATCATCACCTCCGGCAAAGTGAAGC | |
| | TTGCGCGCCACGCACCGGACGGCCGCGAAAACCTG | |
| | CTGACCATCATGGGTCCTTCCGACATGTTCGGTGAG | |
| | CTCTCCATCTTCGACCCAGGCCCACGCACCTCCTCT | |
| | GCAGTGTGTGTCACCGAAGTTCATGCAGCAACCATG | |
| | AACTCTGACATGCTGCGCAACTGGGTAGCTGACCAC | |
| | CCAGCTATCGCTGAGCAGCTCCTGCGCGTTCTGGC | |
| | TCGTCGTCTGCGTCGCACCAACGCTTCCCTGGCTGA | |
| | CCTCATCTTCACCGACGTCCCAGGCCGCGTTGCTAA | |
| | GACCCTTCTGCAGCTGGCTAACCGCTTCGGCACCC | |
| | AAGAAGCTGGCGCGCTGCGCGTGAACCACGACCTC | |
| | ACTCAGGAAGAAATCGCACAGCTCGTCGGTGCTTCC | |
| | CGTGAAACTGTGAATAAGGCTCTTGCAACGTTCGCA | |
| | CACCGTGGCTGGATTCGCCTCGAGGGCAAGTCCGT | |
| | CCTCATTGTGGACACCGAGCATTTGGCACGTCGCG | |
| | CTCGATAA | |

Herein, the term, “corresponding to” refers to an amino acid residue at a position listed in a polypeptide, or an amino acid residue that is similar, identical or homologous to a residue listed in a polypeptide. Confirming the amino acid at the corresponding position may be determining a specific amino acid in a sequence that refers to a specific sequence. “Corresponding region” used herein generally refers to a similar or corresponding position in a related protein or reference protein.

For example, any amino acid sequence is aligned with SEQ ID NO: 1, and based on this, each amino acid residue of the amino acid sequence may be numbered by reference to the numerical position of the amino acid residue of SEQ ID NO: 1 and the corresponding amino acid residue. For example, a sequence alignment algorithm such as that described herein may confirm the position of an amino acid, or a position where a modification such as substitution, insertion or deletion occurs compared to a query sequence (also referred to as a “reference sequence”).

For such alignment, for example, Needleman-Wunsch algorithm (Needleman and Wunsch, 1970, J. Mol. Biol. 48: 443-453), Needle program of EMBOSS package (EMBOSS: The European Molecular Biology Open Software Suite, Rice et al., 2000), Trends Genet. 16: 276-277), and the like may be used, but not limited thereto, and sequence alignment programs, pairwise sequence comparison algorithm, and the like known in the art may be appropriately used.

Herein, that a polynucleotide or polypeptide “comprises a specific nucleic acid sequence (nucleotide sequence) or amino acid sequence or consists of the sequence” may mean that the polynucleotide or polypeptide necessarily comprises the specific nucleic acid sequence (nucleotide sequence) or amino acid sequence, and it may be interpreted as including the “substantially equivalent sequence” in which mutation (deletion, substitution, modification and/or insertion) is added to the specific nucleic acid sequence (nucleotide sequence) or amino acid sequence (or not excluding such mutation) within the range of maintaining the original function and/or desired function of the polynucleotide or polypeptide. In one example, that a polynucleotide or polypeptide “comprises a specific nucleic acid sequence (nucleotide sequence) or amino acid sequence or consists of the sequence” may mean that the polynucleotide or polypeptide (i) necessarily comprises the specific nucleic acid sequence (nucleotide sequence) or amino acid sequence, or (ii) consists of the nucleic acid sequence or amino acid sequence having homology or identity of 70% or more, 80% or more, 85% or more, 90% or more, 91% or more, 92% or more, 93% or more, 94% or more, 95% or more, 96% or more, 97% or more, 98% or more, 98% or more, 99% or more, 99.5% or more, or 99.9% or more to the specific nucleic acid sequence (nucleotide sequence) or amino acid sequence or necessarily comprises that and maintains the original function and/or desired function. In one example, the desired function may mean a function of increasing or giving the L-threonine productivity of a microorganism.

Herein, “homology” means the percent of identity between two polynucleotide or polypeptide moieties. The homology between sequences from one moiety to the other moiety may be determined by known art. For example, homology may be determined by directly aligning sequence information between two polynucleotide molecules or two polypeptide molecules using a readily available computer program aligning sequence information, for example, parameters such as score, identity and similarity, and the like. The computer program may be BLAST (NCBI), CLC Main Workbench (CLC bio), MegAlign™ (DNASTAR Inc), and the like. In addition, the homology between polynucleotides may be determined by hybridizing polynucleotides under conditions of forming a stable double strand between homologous regions, and then degrading them with single-stranded-specific nuclease to confirm the size of the degraded fragment.

Herein, ‘homology or identity’ means the degree of similarity between two given amino acid sequences or nucleotide sequences and may be expressed as a percentage. The terms, homology and identity may be often used interchangeably.

The sequence homology or identity of the conserved polynucleotide or polypeptide is determined by a standard arrangement algorithm, and a default gap penalty established by a used program may be used together. Substantially, homologous or identical sequence may be generally hybridized under moderate or high stringent conditions with all or part of a sequence. It is obvious that hybridization also includes hybridization with a polynucleotide containing a codon in consideration of a general codon or codon degeneracy in a polynucleotide.

Whether any two polynucleotide or polypeptide sequences have homology, similarity or identity may be determined, for example, using a known computer algorithm such as “FASTA” program using a default parameter such as Pearson et al (1988) [Proc. Natl. Acad. Sci. USA 85]: 2444. Otherwise, it may be determined using Needleman-Wunsch algorithm (Needleman and Wunsch, 1970, J. Mol. Biol. 48: 443-453), as performed in the Needleman program of EMBOSS package (EMBOSS: The European Molecular Biology Open Software Suite, Rice et al., 2000, Trends Genet. 16: 276-277) (version 5.0.0 or later version) (including GCG program package (Devereux, J., et al, Nucleic Acids Research 12: 387 (1984)), BLASTP, BLASTN, FASTA (Atschul, [S.][F.,][ET AL, J MOLEC BIOL 215]: 403 (1990); Guide to Huge Computers, Martin J. Bishop, [ED.,] Academic Press, San Diego, 1994, and [CARILLO ETA/.](1988) SIAM J Applied Math 48: 1073). For example, the homology, similarity or identity may be determined using BLAST or ClustalW of National Center for Biotechnology Information.

The homology, similarity or identity of the polynucleotide or polypeptide may be determined by comparing sequence information, for example, using GAP computer program such as Needleman et al. (1970), J Mol Biol. 48:443, for example, known in Smith and Waterman, Adv. Appl. Math (1981) 2:482. In summary, the GAP program may be defined as a value of dividing the number of similarly aligned symbols (i.e., nucleotide or amino acid) by the total number of symbols in the shorter of two sequences. The default parameter for the GAP program may comprise (1) binary comparison matrix (containing values of 1 for identity and 0 for non-identity) and weighted comparison matrix of Gribskov et al (1986) Nucl. Acids Res. 14: 6745 disclosed in Schwartz and Dayhoff, eds., Atlas Of Protein Sequence And Structure, National Biomedical Research Foundation, pp. 353-358 (1979) (or EDNAFULL (EMBOSS version of NCBI NUC4.4) substitution matrix); (2) 3.0 penalty for each gap and additional 0.10 penalty for each symbol in each gap (or gap opening penalty 10, gap extending penalty 0.5); and (3) no penalty for end gaps.

One aspect may provide a GlxR protein variant in which an amino acid at a position of one or more selected from the group consisting of the 45^{th}, 66^{th}, 166^{th} and 168^{th} from the N-terminus in the amino acid sequence of SEQ ID NO: 1 and/or corresponding thereto (to one or more positions selected above) is substituted with other amino acid. The “other amino acid” may mean other amino acid except for amino acids positioned at a position of one or more selected from the group consisting of the 45^{th}, 66^{th}, 166^{th} and 168^{th} of SEQ ID NO: 1 and/or corresponding thereto.

The variant according to one example may comprise a polypeptide in which an amino acid residue at a position of one or more selected from the group consisting of the 45^{th}, 66^{th}, 166^{th} and 168^{th} of the amino acid sequence of SEQ ID NO: 1 and/or corresponding thereto (at one or more positions selected above) is substituted with other kind of amino acid residue in the amino acid sequence having homology or identity of at least 70%, 75%, 80%, 85%, 90%, 95%, 96%, 97%, 98%, 99%, 99.5%, 99.7% or 99.9% or more, to the amino acid sequence represented by SEQ ID NO: 1. In addition, it is obvious that variants having an amino acid sequence in which some sequences are deleted, modified, substituted, conservatively substituted or added are also included within the scope of the present application, if the protein has such homology or identity and has the same or corresponding activity to the GlxR protein.

In one example, a GlxR (glyoxylate bypass regulator) protein variant, comprising a mutation selected from the group consisting of (1) to (4) below:
(1) substitution of the residue corresponding to the 45^{th} amino acid position from the N-terminus in the amino acid sequence of SEQ ID NO: 1 (for example, glutamic acid, or glutamate) with alanine, serine, phenylalanine or lysine;
(2) substitution of the residue corresponding to the 66^{th} amino acid position from the N-terminus in the amino acid sequence of SEQ ID NO: 1 (for example, aspartic acid, or aspartate) with alanine, phenylalanine or lysine;
(3) substitution of the residue corresponding to the 166^{th} amino acid position from the N-terminus in the amino acid sequence of SEQ ID NO: 1 (for example, threonine) with aspartic acid, phenylalanine or lysine; and
(4) substitution of the residue corresponding to the 168^{th} amino acid position from the N-terminus in the amino acid sequence of SEQ ID NO: 1 (for example, glutamic acid, or glutamate) with alanine, phenylalanine or lysine.

In addition, the variant of the present application may comprise an amino acid in which an amino acid residue at a position of one or more selected from the group consisting of the 45^{th}, 66^{th}, 166^{th} and 168^{th} based on the amino acid sequence of SEQ ID NO: 1 and/or corresponding thereto (at one or more positions selected above) is substituted with other kind of amino acid residue, which has homology or identity of at least 70%, 75%, 80%, 85%, 90%, 95%, 96%, 97%, 98%, 99%, 99.5%, 99.7% or 99.9% or more to the amino acid sequence selected from SEQ ID NOs: 3 to 15. Furthermore, it is obvious that variants having an amino acid sequence in which some sequences are deleted, modified, substituted, conservatively substituted or added are also included within the scope of the present application, if the amino acid sequence has such homology or identity and exhibits the corresponding effect to the variant of the present application.

For example, there are cases of having a sequence insertion or deletion which does not alter the function of the variant of the present application at the N-terminus, C-terminus and/or within the amino acid sequence, naturally occurring mutation, silent mutation or conservative substitution.

The “conservative substitution” means substituting an amino acid with another amino acid having similar structural and/or chemical properties. Such amino acid substitution may generally occur based on similarity in the polarity, charge, solubility, hydrophobicity, hydrophilicity and/or amphipathic nature of the residue. Typically, the conservative substitution may have little or no effect on the activity of protein or polypeptide.

Herein, the term “variant” refers to a polypeptide in which one or more amino acids are conservatively substituted and/or modified so as to be different from the amino acid sequence before mutation of the variant, but functions or properties are maintained. Such variant may generally be identified by modifying one or more amino acids in the amino acid sequence of the polypeptide and evaluating the properties of the modified polypeptide. In other words, the ability of the variant may be increased, unchanged or decreased, compared to the polypeptide before mutation. In addition, some variants may include variants in which one or more portions such as an N-terminal leader sequence or a transmembrane domain are removed. Other variants may include variants in which a portion has been removed from the N- and/or C-terminus of the mature protein. The term “variant” may be used interchangeably with terms such as mutant, modification, mutant polypeptide, mutated protein, mutation and variant, and the like (in English, modification, modified polypeptide, modified protein, variant, mutein, divergent, etc.), and as long as it is a term used in a mutated sense, it is not limited thereto. In addition, the variant may comprise deletion or insertion of amino acids having minimal effect on the properties and secondary structure of the polypeptide. For example, a signal (or leader) sequence involved in co-translational or post-translational protein translocation may be conjugated at the N-terminus of the variant. Furthermore, the variant may be conjugated with other sequences or linkers for identification, purification or synthesis.

According to one example, the GlxR protein variant may comprise the amino acid sequence selected from the group consisting of SEQ ID NO: 3 to SEQ ID NO: 15 or consist of the amino acid sequence. The amino acid sequences of SEQ ID NO: 3 to SEQ ID NO: 15 are described in Table 2 below.

TABLE 2

| Name | Sequence | SEQ ID NO |
|---|---|---|
| GlxR_E45A | MEGVQEILSRAGIFQGVDPTAVNNLIQDMETVRFPRGA | SEQ ID NO: 3 |
| | TIFDEGAPGDRLYIITSGKVKLARHAPDGRENLLTIMGP | |
| | SDMFGELSIFDPGPRTSSAVCVTEVHAATMNSDMLRN | |
| | WVADHPAIAEQLLRVLARRLRRTNASLADLIFTDVPGR | |
| | VAKTLLQLANRFGTQEAGALRVNHDLTQEEIAQLVGAS | |
| | RETVNKALATFAHRGWIRLEGKSVLIVDTEHLARRAR | |
| GlxR_E45S | MEGVQEILSRAGIFQGVDPTAVNNLIQDMETVRFPRGA | SEQ ID NO: 4 |
| | TIFDEGSPGDRLYIITSGKVKLARHAPDGRENLLTIMGP | |
| | SDMFGELSIFDPGPRTSSAVCVTEVHAATMNSDMLRN | |
| | WVADHPAIAEQLLRVLARRLRRTNASLADLIFTDVPGR | |
| | VAKTLLQLANRFGTQEAGALRVNHDLTQEEIAQLVGAS | |
| | RETVNKALATFAHRGWIRLEGKSVLIVDTEHLARRAR | |
| GlxR_E45F | MEGVQEILSRAGIFQGVDPTAVNNLIQDMETVRFPRGA | SEQ ID NO: 5 |
| | TIFDEGFPGDRLYIITSGKVKLARHAPDGRENLLTIMGP | |
| | SDMFGELSIFDPGPRTSSAVCVTEVHAATMNSDMLRN | |
| | WVADHPAIAEQLLRVLARRLRRTNASLADLIFTDVPGR | |
| | VAKTLLQLANRFGTQEAGALRVNHDLTQEEIAQLVGAS | |
| | RETVNKALATFAHRGWIRLEGKSVLIVDTEHLARRAR | |
| GlxR_E45K | MEGVQEILSRAGIFQGVDPTAVNNLIQDMETVRFPRGA | SEQ ID NO: 6 |
| | TIFDEGKPGDRLYIITSGKVKLARHAPDGRENLLTIMGP | |
| | SDMFGELSIFDPGPRTSSAVCVTEVHAATMNSDMLRN | |
| | WVADHPAIAEQLLRVLARRLRRTNASLADLIFTDVPGR | |
| | VAKTLLQLANRFGTQEAGALRVNHDLTQEEIAQLVGAS | |
| | RETVNKALATFAHRGWIRLEGKSVLIVDTEHLARRAR | |
| GlxR_D66A | MEGVQEILSRAGIFQGVDPTAVNNLIQDMETVRFPRGA | SEQ ID NO: 7 |
| | TIFDEGEPGDRLYIITSGKVKLARHAPAGRENLLTIMGP | |
| | SDMFGELSIFDPGPRTSSAVCVTEVHAATMNSDMLRN | |
| | WVADHPAIAEQLLRVLARRLRRTNASLADLIFTDVPGR | |
| | VAKTLLQLANRFGTQEAGALRVNHDLTQEEIAQLVGAS | |
| | RETVNKALATFAHRGWIRLEGKSVLIVDTEHLARRAR | |
| GlxR_D66F | MEGVQEILSRAGIFQGVDPTAVNNLIQDMETVRFPRGA | SEQ ID NO: 8 |
| | TIFDEGEPGDRLYIITSGKVKLARHAPFGRENLLTIMGP | |
| | SDMFGELSIFDPGPRTSSAVCVTEVHAATMNSDMLRN | |
| | WVADHPAIAEQLLRVLARRLRRTNASLADLIFTDVPGR | |
| | VAKTLLQLANRFGTQEAGALRVNHDLTQEEIAQLVGAS | |
| | RETVNKALATFAHRGWIRLEGKSVLIVDTEHLARRAR | |
| GlxR_D66K | MEGVQEILSRAGIFQGVDPTAVNNLIQDMETVRFPRGA | SEQ ID NO: 9 |
| | TIFDEGEPGDRLYIITSGKVKLARHAPKGRENLLTIMGP | |
| | SDMFGELSIFDPGPRTSSAVCVTEVHAATMNSDMLRN | |
| | WVADHPAIAEQLLRVLARRLRRTNASLADLIFTDVPGR | |
| | VAKTLLQLANRFGTQEAGALRVNHDLTQEEIAQLVGAS | |
| | RETVNKALATFAHRGWIRLEGKSVLIVDTEHLARRAR | |
| GlxR_T166D | MEGVQEILSRAGIFQGVDPTAVNNLIQDMETVRFPRGA | SEQ ID NO: 10 |
| | TIFDEGEPGDRLYIITSGKVKLARHAPDGRENLLTIMGP | |
| | SDMFGELSIFDPGPRTSSAVCVTEVHAATMNSDMLRN | |
| | WVADHPAIAEQLLRVLARRLRRTNASLADLIFTDVPGR | |
| | VAKTLLQLANRFGDQEAGALRVNHDLTQEEIAQLVGAS | |
| | RETVNKALATFAHRGWIRLEGKSVLIVDTEHLARRAR | |
| GlxR_T166F | MEGVQEILSRAGIFQGVDPTAVNNLIQDMETVRFPRGA | SEQ ID NO: 11 |
| | TIFDEGEPGDRLYIITSGKVKLARHAPDGRENLLTIMGP | |
| | SDMFGELSIFDPGPRTSSAVCVTEVHAATMNSDMLRN | |
| | WVADHPAIAEQLLRVLARRLRRTNASLADLIFTDVPGR | |
| | VAKTLLQLANRFGFQEAGALRVNHDLTQEEIAQLVGAS | |
| | RETVNKALATFAHRGWIRLEGKSVLIVDTEHLARRAR | |
| GlxR_T166K | MEGVQEILSRAGIFQGVDPTAVNNLIQDMETVRFPRGA | SEQ ID NO: 12 |
| | TIFDEGEPGDRLYIITSGKVKLARHAPDGRENLLTIMGP | |
| | SDMFGELSIFDPGPRTSSAVCVTEVHAATMNSDMLRN | |
| | WVADHPAIAEQLLRVLARRLRRTNASLADLIFTDVPGR | |
| | VAKTLLQLANRFGKQEAGALRVNHDLTQEEIAQLVGAS | |
| | RETVNKALATFAHRGWIRLEGKSVLIVDTEHLARRAR | |
| GlxR_E168A | MEGVQEILSRAGIFQGVDPTAVNNLIQDMETVRFPRGA | SEQ ID NO: 13 |
| | TIFDEGEPGDRLYIITSGKVKLARHAPDGRENLLTIMGP | |
| | SDMFGELSIFDPGPRTSSAVCVTEVHAATMNSDMLRN | |
| | WVADHPAIAEQLLRVLARRLRRTNASLADLIFTDVPGR | |
| | VAKTLLQLANRFGTQAAGALRVNHDLTQEEIAQLVGAS | |
| | RETVNKALATFAHRGWIRLEGKSVLIVDTEHLARRAR | |
| GlxR_E168F | MEGVQEILSRAGIFQGVDPTAVNNLIQDMETVRFPRGA | SEQ ID NO: 14 |
| | TIFDEGEPGDRLYIITSGKVKLARHAPDGRENLLTIMGP | |
| | SDMFGELSIFDPGPRTSSAVCVTEVHAATMNSDMLRN | |
| | WVADHPAIAEQLLRVLARRLRRTNASLADLIFTDVPGR | |
| | VAKTLLQLANRFGTQFAGALRVNHDLTQEEIAQLVGAS | |
| | RETVNKALATFAHRGWIRLEGKSVLIVDTEHLARRAR | |
| GlxR_E168K | MEGVQEILSRAGIFQGVDPTAVNNLIQDMETVRFPRGA | SEQ ID NO: 15 |
| | TIFDEGEPGDRLYIITSGKVKLARHAPDGRENLLTIMGP | |
| | SDMFGELSIFDPGPRTSSAVCVTEVHAATMNSDMLRN | |
| | WVADHPAIAEQLLRVLARRLRRTNASLADLIFTDVPGR | |
| | VAKTLLQLANRFGTQKAGALRVNHDLTQEEIAQLVGAS | |
| | RETVNKALATFAHRGWIRLEGKSVLIVDTEHLARRAR | |

The GlxR protein variant according to one example is a global transcriptional regulator, as GlxR protein before mutation (for example, GlxR protein consisting of the amino acid sequence of SEQ ID NO: 1), and for example, it may have the function of the regulator for glyoxylate bypass genes (for example, ace A and/or ace B).

The GlxR protein variant according to one example may have a weakened activity than the GlxR protein (a wildtype protein (for example, GlxR protein consisting of the amino acid sequence of SEQ ID NO: 1)) before mutation. The “weakened activity of the GlxR protein” may mean that the level of activity of the global transcriptional regulator in the cell is reduced compared to the protein before mutation. The weakening is when the activity of the protein itself is reduced compared to the activity of the protein possessed by the original microorganism (for example, wildtype, parent strain, host microorganism, unmodified microorganism, etc.) due to mutation of the gene encoding GlxR protein, and when the expression level of the enzyme is lowered due to inhibition of transcription and/or inhibition of translation of the gene encoding GlxR protein, and thus the overall degree of enzymatic activity in the cell is low, a combination thereof may also be included.

Herein, the GlxR protein variant may be used as the same meaning as mutant GlxR protein, GlxR variant and mutant GlxR.

Another aspect may provide a polynucleotide, encoding the GlxR protein variant.

Herein, “polynucleotide” refers to a DNA or RNA strand of a certain length or longer as a polymer of nucleotides in which nucleotide monomers are connected in a long chain form by covalent bonds, and more specifically, a polynucleotide fragment encoding the variant.

The polynucleotide may be included without limitation, as long as it is a polynucleotide sequence encoding the GlxR protein variant according to one example. The polynucleotide according to one example may comprise a nucleic acid sequence encoding a GlxR protein variant in which an amino acid at a position of one or more selected from the group consisting of the 45^{th}, 66^{th}, 166^{th} and 168^{th}, from the N-terminus in the amino acid sequence of SEQ ID NO: 1 and/or corresponding thereto (at one or more positions selected above), or consist of the nucleic acid sequence, and for example, it may comprise a nucleic acid sequence encoding the amino acid sequence selected from the group consisting of SEQ ID NO: 3 to SEQ ID NO: 15 or consist of the nucleic acid sequence.

The polynucleotide may have various modifications in the coding region within a range which does not change the amino acid sequence of the variant according to one example, considering codon degeneracy or preferred codons in organisms that want to express the variant according to one example. Accordingly, by codon degeneracy, the polypeptide consisting of the amino acid sequence of SEQ ID NO: 3 to SEQ ID NO: 15 or a polynucleotide capable of encoding a polypeptide having homology (or identity) thereto may also be included.

The polynucleotide according to one example may be included without limitation, as long as it is a probe that may be prepared from a known gene sequence, for example, a sequence capable of hybridizing under a stringent condition with a complementary sequence to all or part o the polynucleotide according to one example. The “stringent condition” means a condition that enables specific hybridization between polynucleotides.

This condition is specifically described in the document (See J. Sambrook et al., Molecular Cloning, A Laboratory Manual, 2nd Edition, Cold Spring Harbor Laboratory press, Cold Spring Harbor, New York, 1989; F. M. Ausubel et al., Current Protocols in Molecular Biology, John Wiley & Sons, Inc., New York, 9.50-9.51, 11.7-11.8). For example, a condition in which polynucleotides with high homology or identity, polynucleotides with homology or identity of 70% or more, 75% or more, 80% or more, 85% or more, 90% or more, 95% or more, 96% or more, 97% or more, 98% or more, or 99% or more, are hybridized, and polynucleotides with low homology or identity are not hybridized, or a condition for washing once, specifically, twice or 3 times, at a salt concentration and temperature corresponding to a common southern hybridization washing condition of 60° C., 1×SSC, 0.1% SDS, specifically, 60° C., 0.1×SSC, 0.1% SDS, more specifically, 68° C., 0.1×SSC, 0.1% SDS.

Hybridization requires that two nucleic acids have complementary sequences, although mismatch between bases is possible depending on the stringency of hybridization. Herein, “complementary” is used to describe the relationship between nucleotide bases capable of hybridizing to each other. For example, with respect to DNA, adenine is complementary to thymine and cytosine is complementary to guanine. Thus, the polynucleotide according to one example may also comprise an isolated nucleic acid fragment complementary to the overall sequence as well as substantially similar nucleic acid sequences.

Specifically, the polynucleotide having homology or identity to the polynucleotide according to one example may be detected using a hybridization condition including hybridizing at a Tm value of 50° C. to 65° C. (for example, 55° C.) using the aforementioned condition. In addition, the Tm value may be 60° C., 63° C. or 65° C., but not limited thereto, and it may be appropriately adjusted by those skilled in the art according to the purpose.

The appropriate stringency for hybridizing the polynucleotide depends on the length and degree of complementarity of the polynucleotide, and the parameters are well known in the art (for example, see J. J. Sambrook et al., Molecular Cloning, A Laboratory Manual, 2nd Edition, Cold Spring Harbor Laboratory press, Cold Spring Harbor, New York, 1989; F. M. Ausubel et al., Current Protocols in Molecular Biology, John Wiley & Sons, Inc., New York, 9.50-9.51, 11.7-11.8).

Other aspect may provide a vector, comprising the polynucleotide. The vector may be an expression vector for expressing the polynucleotide in a host cell, but not limited thereto.

Herein, “vector” may comprise a DNA product comprising a nucleotide sequence of a polynucleotide encoding the target polypeptide operably linked to a suitable expression control region (or expression control sequence) so as to express a target polypeptide in a suitable host. the expression control region may comprise a promoter capable of initiating transcription, any operator sequence for regulating such transcription, a sequence encoding a suitable mRNA ribosome binding site, and a sequence regulating termination of transcription and translation. The vector may be replicated or function independently of the host genome and may be integrated into the genome itself, after being transformed into an appropriate host cell.

The vector used herein is not particularly limited, and any vector known in the art may be used. The example of the commonly used vector may include a natural or recombinant plasmid, a cosmid, a virus and a bacteriophage. For example, as a phage vector or cosmid vector, pWE15, M13, MBL3, MBL4, IXII, ASHII, APII, t10, t11, Charon4A, and Charon21A, and the like may be used, and as a plasmid vector, pDZ-based, pBR-based, pUC-based, pBluescriptII-based, pGEM-based, pTZ-based, pCL-based and pET-based, and the like may be used. Specifically, pDZ, pDC, pDCM2, pACYC177, pACYC184, pCL, pECCG117, pUC19, pBR322, pMW118, pCC1BAC vectors, and the like may be used.

In one example, a polynucleotide encoding a protein (for example, GlxR protein) in chromosome may be replaced with a mutated polynucleotide through a vector for intracellular chromosome insertion. the insertion into the chromosome of the polynucleotide may be achieved by any method known in the art, for example, homologous recombination, but not limited thereto. A selection marker for confirming the chromosome insertion may be further comprised. The selection marker is used to select cells transformed with the vector, that is, to confirm whether a target polynucleotide molecule is inserted, and markers conferring selectable phenotypes such as drug resistance, auxotrophy, resistance to cytotoxic agents or expression of surface mutant protein may be used. In an environment treated with a selective agent, only the cells expressing the selectable marker survive or exhibit other expression traits, so that the transformed cells may be selected.

Herein, “transformation” means introducing a vector comprising a polynucleotide encoding a target polynucleotide into a host cell or microorganism so that the polypeptide encoded by the polynucleotide may be expressed in the host cell. the transformed polynucleotide may include all of them regardless of whether they are inserted into the chromosome of the host cell or located outside the chromosome, as long as they can be expressed in the host cell. In addition, the polynucleotide comprises DNA and/or RNA encoding a target polypeptide (for example, the GlxR variant). The polynucleotide may be introduced in any form as long as it may be introduced and expressed into a host cell. For example, the polynucleotide may be introduced into a host cell in the form of an expression cassette, which is a gene construct including all elements necessary for self-expression. the expression cassette may comprise a promoter, a transcription termination signal, a ribosome binding site and a translation termination signal, which are usually operably linked to the polynucleotide. The expression cassette may be in the form of an expression vector capable of self-replication. In addition, the polynucleotide may be introduced into a host cell in its own form and operably linked to a sequence required for expression in the host cell, but is not limited thereto. The transformation method is included without limitation as long as it is a method of introducing a gene into a cell, and may be performed by selecting a suitable standard technique known in the art depending on the host cell. For example, electroporation, calcium phosphate (CaPO₄) precipitation, calcium chloride (CaCl₂)) precipitation, microinjection, retroviral infection, polyethylene glycol (PEG) method, DEAE-dextran method, cation liposome method, and/or lithium acetate-DMSO method, and the like may be used, but not limited thereto.

The vector may be a DNA product containing a nucleic acid sequence of a polynucleotide encoding the target protein operably linked to a suitable regulatory sequence to allow expression of the target protein (for example, the GlxR variant) in a suitable host. The regulatory sequence may include a promoter capable of initiating transcription, any operator sequence for regulating such transcription, a sequence encoding a suitable mRNA ribosome binding site and a sequence regulating termination of transcription and translation. The vector may be replicated or function independently of the host genome and may be integrated into the genome itself, after being transformed into an appropriate host cell.

Herein, “operably linked” means that the polynucleotide sequence is functionally linked to a promoter sequence that initiates and mediates transcription of the polynucleotide encoding the target variant of the present application.

Other aspect may provide a microorganism (or, strain, recombinant cell), comprising one or more kinds selected from the group consisting of the GlxR protein variant, a polynucleotide encoding the GlxR protein variant, and a vector comprising the polynucleotide. Herein, “microorganism” may include one-celled bacteria.

Herein, “strain (or, microorganism)” includes both wildtype microorganisms and microorganisms that have undergone natural or artificial genetic modification, and is a microorganism in which a specific mechanism is weakened or enhanced due to causes such as insertion of an external gene or intensification or inactivation of the activity of an endogenous gene, which is a microorganism comprising a genetic modification for production of a desired polypeptide, protein or product.

The microorganism (or, strain, recombinant cell) may be a strain comprising one or more selected from the GlxR protein variant, polynucleotide according to one example and vector comprising the polynucleotide according to one example; a strain modified to express the GlxR protein variant or polynucleotide according to one example; a strain (for example, recombinant strain) expressing the GlxR protein variant or polynucleotide according to one example; or a strain (for example, recombinant strain) having the GlxR protein variant activity, but not limited thereto.

In one example, the microorganism (or, strain, recombinant cell) may have the threonine (for example, L-threonine) productivity. The microorganism (or, strain, recombinant cell) may have the enhanced threonine productivity than a non-modified microorganism, a cell before recombination, a parent strain and/or a wildtype strain, or may be given the threonine productivity different from the non-modified microorganism, a cell before recombination, a parent strain and/or a wildtype strain, which have no threonine productivity.

Herein, “have threonine (for example, L-threonine) productivity” means a cell and/or microorganism naturally having the threonine productivity, or a microorganism in which the threonine productivity is given to a parent strain having no threonine productivity. It may be used to mean the case that the microorganism has the L-threonine productivity by introducing the GlxR protein variant according to one example and/or the case that the microorganism has the L-threonine productivity by introducing the GlxR protein variant into a microorganism having no L-threonine productivity. For example, a Corynebacterium sp. microorganism having the L-threonine productivity may mean a native microorganism itself or a Corynebacterium sp. microorganism having the enhanced L-threonine productivity in which an external gene related to threonine production mechanism is inserted or the activity of an intrinsic gene is strengthened, or weakened or inactivated.

The microorganism in which the GlxR protein mutation according to one example is introduced may have increased threonine (for example, L-threonine), compared to the same kind of non-modified microorganism. Herein, “non-modified microorganism” does not exclude strains comprising a mutation which can occur naturally in microorganisms, and may mean a wildtype strain or natural-type strain itself, or a strain before the trait is changed due to genetic mutation caused by natural or artificial factors. For example, the non-modified microorganism may mean a strain in which the GlxR variant according to one example is not introduced or before introduction. The “non-modified microorganism” may be interchangeably used with “strain before modification”, “microorganism before modification”, “non-mutated strain”, “non-modified strain”, “non-mutated microorganism” or “standard microorganism”. That the GlxR protein mutation is introduced into a microorganism above may mean that the GlxR protein variant, a polynucleotide encoding the GlxR protein variant and/or a vector comprising the polynucleotide. In one example, the protein non-modified microorganism, which is a subject strain comparing the increase of the threonine productivity may be ATCC13032 strain, KCCM12000P strain, and/or KCCM12120P strain, but not limited thereto.

In one example, the recombinant strain with increased productivity may have increased threonine productivity of about 1% or more, about 2.5% or more, about 5% or more, about 6% or more, about 7% or more, about 8% or more, about 9% or more, about 10% or more, about 10.5% or more, about 11% or more, about 11.5% or more, about 12% or more, about 12.5% or more, about 13% or more, about 13.5% or more, about 14% or more, about 14.5% or more, about 15% or more, about 15.5% or more, about 16% or more, about 16.5% or more, about 17% or more, about 17.5% or more, about 18% or more, about 18.5% or more, about 19% or more, about 19.5% or more, about 20% or more, about 20.5% or more, about 21% or more, about 21.5% or more, about 22% or more, about 22.5% or more, about 23% or more, about 23.5% or more, about 24% or more, about 24.5% or more, about 25% or more, about 25.5% or more, about 26% or more, about 26.5% or more, about 27% or more, (the upper limit is not particularly limited, and for example, about 200% or less, about 150% or less, about 100% or less, about 50% or less, about 45% or less, about 40% or less, or about 35% or less), compared to the parent strain before mutation or non-modified microorganism. In other example, the recombinant strain with increased productivity may have increased threonine productivity of about 1.1 times or more, about 1.12 times or more, about 1.13 times or more, 1.15 times or more, 1.16 times or more, 1.17 times or more, 1.18 times or more, 1.19 times or more, about 1.2 times or more, about 1.25 times or more, about 1.26 times or more, or about 1.3 times or more (the upper limit is not particularly limited, and for example, about 10 times or less, about 5 times or less, about 3 times or less, or about 2 times or less), compared to the parent strain before mutation or non-modified microorganism. In other example, the recombinant strain with increased productivity may have increased threonine productivity of about 1.1 times or more, about 1.12 times or more, compared to the parent strain before mutation or non-modified microorganism. The term, “about” is a range including all of ±0.5, ±0.4, ±0.3, ±0.2, ±0.1, and the like, and it includes all numerical values within a range equal or similar to the numerical value following the term about.

The insertion of the polynucleotide into host cell genome (chromosome) or introduction of mutation so that a host cell endogenous gene (for example, gIxR gene) encodes a GlxR protein variant may be performed by appropriately selecting a known method by those skilled in the art, and for example, it may be performed using an RNA-guided endonuclease system; for example, (a) RNA-guided endonuclease (e.g., Cas9 protein, etc.), its encoding gene, or a vector comprising the gene; and (b) guided RNA (e.g., single guide RNA (sgRNA), etc.), its encoding DNA, or a mixture comprising a vector comprising the DNA (for example, a mixture of the RNA-guided endonuclease protein and guided RNA, etc.), or a complex (for example, one or more selected from the group consisting of ribonucleic acid fusion protein (RNP), a recombinant vector (for example, a vector comprising the RNA-guided endonuclease encoding gene and guided RNA encoding DNA together, etc.), and the like), but not limited thereto. Modification of a part or all of a polynucleotide in the microorganism according to one example (for example, modification for encoding the aforementioned protein variant) may be induced by (a) homologous recombination using a vector for insertion of chromosome into a microorganism or genome editing using a gene scissor (engineered nuclease, e.g., CRISPR-Cas9) and/or (b) light and/or chemical substance treatment such as ultraviolet and radiation, and the like, but not limited thereto. The method for modifying part or all of the gene may include a method by DNA recombination technology. For example, a part or all of the gene may be deleted by injecting a nucleotide sequence or vector comprising a nucleotide sequence having homology to a target gene to cause homologous recombination. The nucleotide sequence or vector to be injected may comprise a dominant selectable marker, but not limited thereto.

The microorganism (or strain, recombinant cell) may further comprise mutation to increase threonine production, and the position of the mutation and/or kind of the gene and/or protein to be mutated may be comprised without limitation as long as it increases threonine production. The recombinant cell may be used without limitation as long as it is a transformable cell.

In order to distinguish from a microorganism in which the threonine productivity is increased or the threonine productivity is given by introducing the GlxR protein mutation, the microorganism before introducing the GlxR protein mutation may be represented by a host microorganism (or parent strain, non-modified microorganism).

In one example, the microorganism comprising the GlxR protein variant, a polynucleotide encoding the variant, and/or a vector comprising the polynucleotide may,
(i) further comprise the polynucleotide and/or the recombinant vector, in addition to genome, and/or
(ii) comprise the polynucleotide, as an endogenous GlxR protein encoding gene (for example, gIxR gene).

The (ii) “comprising the polynucleotide, as an endogenous GlxR protein encoding gene (for example, gIxR gene)” may mean (a) that the polynucleotide is comprised (inserted) by replacing the endogenous GlxR protein encoding gene (for example, gIxR gene), and/or (b) that the endogenous GlxR protein encoding gene (for example, gIxR gene) is mutated to have the nucleic acid sequence of the polynucleotide (that is, nucleic acid sequence encoding GlxR protein variant) by gene editing technology.

In one example, the GlxR protein or a gene encoding the same may be derived from a host microorganism (intrinsic) or derived from a different microorganism (foreign).

In one example, in the microorganism (or strain, recombinant cell), the polynucleotide according to one example may be integrated in the chromosome, and for example, the polynucleotide according to one example may be replaced for the native gene at the gIxR gene site in the chromosome or be integrated at the additional gene site.

The microorganism may be a Corynebacterium sp. microorganism.

The Corynebacterium sp. microorganism may include Corynebacterium glutamicum, Corynebacterium ammoniagenes, Corynebacterium acetoacidophilum, Corynebacterium acetoglutamicum, Corynebacterium alkanolyticum, Corynebacterium callunae, Corynebacterium lilium, Corynebacterium melassecola, Corynebacterium thermoaminogenes, Corynebacterium efficiens, Corynebacterium herculis, Corynebacterium crudilactis, Corynebacterium deserti, Corynebacterium stationis, Corynebacterium singulare, Corynebacterium halotolerans, Corynebacterium striatum, Corynebacterium ammoniagenes, Corynebacterium pollutisoli, Corynebacterium imitans, Corynebacterium testudinoris, and/or Corynebacterium flavescens, and the like.

The microorganism (or strain, recombinant cell) may include both one artificially prepared by transformation, or naturally occurring one. For example, the microorganism (or strain, recombinant cell) may be transformed with a vector comprising a polynucleotide encoding the GlxR protein variant according to one example.

The microorganism (or strain, recombinant cell) may be one in which the polynucleotide sequence according to one embodiment is integrated in the chromosome. Homologous recombination allows, in conjunction with the use of the vector according to one example, the exchange of DNA fragments on the chromosome for the polynucleotide according to one example to be delivered into a cell by the vector. For efficient recombination between circular DNA molecules of the vector and target DNA on the chromosome, in the DNA region to be exchanged containing the polynucleotide according to one example, the nucleotide sequence homologous to the target site at the end is provided, and they determine the site of integration of the vector and exchange of DNA. For example, the polynucleotide according to one example may be exchanged for a native gIxR gene at a natural gene site in the chromosome or may be integrated at an additional gene site.

Other aspect may provide a method for production of threonine, comprising culturing the microorganism (or strain, recombinant cell) in a medium. The threonine may be L-threonine. The microorganism (or strain, recombinant cell) is as described above.

Herein, “culture” refers to growing the microorganism (or strain, recombinant cell) in an appropriately controlled environmental condition. The culture may be made according to an appropriate medium and culture conditions known in the art, and it should satisfy requirements of a specific strain in an appropriate manner, and may be appropriately modified by those skilled in the art. The culture method may include for example, batch culture, continuous culture, fed-batch culture, or a combination thereof, but not limited thereto.

Herein, “medium” means a substance in which nutrients required for culturing the Corynebacterium glutamicum strain of the present application as a main component, and supplies nutrients and growth factors, and the like, including water which is essential for survival and growth. Specifically, the medium and other culture conditions used for culture of the microorganism according to one example may be used without particular limitation as long as it is a medium used for common culture of a microorganism, but the microorganism according to one example may be cultured while adjusting the temperature, pH, and the like under an aerobic condition in a conventional medium containing a suitable carbon source, a nitrogen source, a phosphorus source, an inorganic compound, an amino acid and/or vitamin, and the like.

In one example, the medium for culturing the microorganism (or strain, recombinant cell) may refer to the document [“Manual of Methods for General Bacteriology” by the American Society for Bacteriology (Washington D.C., USA, 1981)], but not limited thereto.

As the carbon source, carbohydrates such as glucose, saccharose, lactose, fructose, sucrose and maltose; sugar alcohols such as mannitol and sorbitol; organic acids such as pyruvate, lactate and citrate; and amino acids such as glutamic acid, methionine and lysine, and the like may be included. In addition, natural organic nutrients such as starch hydrolysate, molasses, blackstrap molasses, rice bran, cassava, sugar cane residues and corn steep liquid may be used, and specifically, carbohydrates such as glucose and sterilized pretreated molasses (that is, molasses converted to reduced sugar), and the like may be used, and other suitable amount of carbon sources may be variously used without limitation. These carbon sources may be used alone or 2 kinds or more may be used in combination, but not limited thereto.

As the nitrogen source, inorganic nitrogen sources such as ammonia, ammonium sulfate, ammonium citrate, ammonium phosphate, ammonium carbonate, ammonium nitrate, and the like; and organic nitrogen sources including amino acids such as glutamic acid, methionine, glutamine, peptone, NZ-amine, meat extract, yeast extract, malt extract, corn steep liquid, casein hydrolysate, fish or its decomposition product, defatted soybean cake or its decomposition product may be used. These nitrogen sources may be used alone or two kinds or more may be used in combination, but not limited thereto.

As the phosphorus source, potassium phosphate monobasic, potassium phosphate dibasic, or sodium-containing salts corresponding thereto, or the like may be included. As the inorganic compound, sodium chloride, calcium chloride, iron chloride, magnesium sulfate, iron sulfate, manganese sulfate, calcium carbonate, and the like may be used, and in addition, amino acids, vitamins and/or appropriate precursors, and the like may be included. These components or precursors may be added to the medium in a batch or continuous manner. However, it is not limited thereto.

Furthermore, the medium may contain a metal salt such as magnesium sulfate or iron sulfate needed for growth, but not limited thereto. In addition, essential growth materials such as amino acids and vitamins may be comprised. Moreover, precursors suitable for the medium may be used. The medium or individual component may be added in a batch or continuous manner by an appropriate method in a culture solution during the culture process, but not limited thereto.

According to one example, a compound such as ammonium hydroxide, potassium hydroxide, ammonia, phosphate, sulfate, and the like may be added to the medium during the culture in an appropriate manner, to adjust the pH of the medium. In addition, during culturing, an antifoaming agent such as fatty acid polyglycol ester may be used to inhibit bubble formation. Furthermore, in order to maintain the aerobic state of the medium, oxygen or oxygen-containing gas may be injected into the medium, or in order to maintain the anaerobic and microaerobic state, gas is not injected or nitrogen, hydrogen or carbon dioxide gas may be injected, but not limited thereto.

According to one example, the culture temperature may be maintained as 20° C. to 45° C., 25° C. to 40° C., or 30° C. to 37° C. The culture period may be continued until a useful substance (for example, L-threonine) is obtained in a desired amount, and for example, it may be 10 to 160 hours.

The production method of threonine according to one example may further comprise preparing the microorganism (or strain, recombinant cell) according to one example, preparing a medium for culturing the microorganism, or a combination thereof (in any order), for example, before the culturing.

The production method of threonine according to one example may further comprise isolating and/or recovering threonine form the cultured medium (culture medium) and/or microorganism (or strain, recombinant cell). The isolating and/or recovering may be further comprised after the culturing. The culture medium may refer to a medium culturing the microorganism (or strain, recombinant cell).

The isolating and/or recovering threonine may collect a desired amino acid (for example, L-threonine) from a medium, culture solution or microorganism using an appropriate method known in the art depending on the culture method (for example, batch, continuous or fed-batch culture method, etc.). For example, a method such as centrifugation, filtration, treatment by a crystallized protein precipitating agent (salting out method), extraction, spraying, drying, evaporation, precipitation, crystallization, ultrasonic disintegration, ultrafiltration, dialysis, electrophoresis, fractional dissolution (for example, ammonium sulfate precipitation), HPLC, and/or chromatography (for example, ion exchange, affinity, hydrophobicity, liquid and size exclusion), and the like may be used, but not limited thereto.

The production method of threonine according to one example may further comprise purifying. The purifying may be performed using an appropriate method known in the art. In one example, when the production method of threonine comprises both isolating and/or recovering and purifying, purifying threonine may be additionally comprised before or after the isolating and/or recovering, or the isolating and/or recovering and purifying may be performed at different times (or continuously) in any order, or may be performed simultaneously or by being integrated into one step, but not limited thereto.

Other aspect may provide a composition for producing threonine (for example, L-threonine), comprising one or more kinds selected from the group consisting of the GlxR protein variant, a polynucleotide encoding the GlxR protein variant, a vector comprising the polynucleotide and a microorganism (or strain, recombinant cell) comprising the polynucleotide or vector. The variant, polynucleotide, vector, microorganism, medium and threonine, and the like are as described above.

In one example, the composition for production may further comprise any appropriate excipient commonly used for a composition for producing an amino acid, and such excipient may be for example, a preservative, wetting agent, dispersant, suspending agent, buffer, stabilizer or tonicifying agent, or the like, but not limited thereto.

Other aspect may provide a method of increasing threonine (for example, L-threonine) productivity of the microorganism or a method of giving threonine (for example, L-threonine) productivity to the microorganism, comprising introducing (for example, transforming) the GlxR protein variant, a polynucleotide encoding the same and/or a recombinant vector comprising the polynucleotide into a microorganism.

### Advantageous Effects

The GlxR protein variant according to one example is introduced into a microorganism, and thereby, it may reduce the production of by-products and significantly increase the productivity of threonine.

### MODE FOR INVENTION

Hereinafter, the present application will be described in more detail by examples and experimental examples. However, these examples and experimental examples are intended to illustrate the present application exemplarily, but the scope of the present application is not limited by these examples and experimental examples.

### Example 1: Construction of Vector Library for Introducing Mutation in gIxR Gene ORF

In order to discover a variant in which the expression of GlxR protein of Corynebacterium glutamicum (SEQ ID NO: 1) or its activity was changed, a vector library was constructed by the following method.

At first, primers of SEQ ID NO: 16 and SEQ ID NO: 17, in which a restriction enzyme SmaI recognition site was inserted into the 5′ fragment and 3′ fragment at the position about 1000 bp away, back and forth, respectively, from the position of 133 to 135^{th} 196 to 198^{th} 496 to 498^{th}, and 502 to 504^{th} in the nucleotide sequence (SEQ ID NO: 2) of the gIxR gene using the genomic DNA extracted from the WT strain (ATCC13032) as a template.

Specifically, DNA fragments positioned at the 5′ and 3′ terminuses of the gIxR gene (1000 bp each) were constructed in a form connected to the pDZ vector (Korean Patent Publication No. 10-2008-0025355). The 5′ terminal gene fragment using primers of SEQ ID NOs: 16 and 18 and the 3′ terminal gene fragment using primers of SEQ ID NOs: 17 and 19 were constructed through PCR using chromosome of the WT strain as a template. PCR conditions were repeating denaturation at 94° C. for 5 minutes, denaturation at 94° C. for 30 seconds, annealing at 55° C. for 30 seconds, and polymerization at 72° C. for 1 minute, 24 times, and then performing polymerization at 72° C. for 10 minutes. After purifying the amplified DNA fragment using Quiagen's PCR Purification kit, it was used as an insertion DNA fragment for vector construction.

On the other hand, the pDZ vector treated with the restriction enzyme SmaI and then heat-treated at 65° C. for 60 minutes and the insertion DNA fragment amplified through PCR were connected using Infusion Cloning Kit, and then transformed into E. coli DH5α. The strain was plated on LB solid medium containing kanamycin (25 mg/l). After selecting colonies transformed with the vector into which the desired gene was inserted through PCR using primers of SEQ ID NOs: 20 and 21, a plasmid was obtained using a commonly known plasmid extraction method. The plasmid was named pDZ-gIxR (E45A).

By the same method, pDZ-gIxR (D66A) using primers of SEQ ID NOs: 16 and 28, 17 and 29, pDZ-gIxR (T166D) using primers of SEQ ID NOs: 16 and 34, 17 and 35, and pDZ-gIxR (E168A) using primers of SEQ ID NOs: 16 and 40, 17 and 41 were constructed.

The nucleic acid sequences of the primers used in the present example were described in Table 3 below.

TABLE 3

| Name | Sequence (5′→3′) | SEQ ID NO |
|---|---|---|
| glxR L1 | GAATTCGAGCTCGGTACCCTGAGACCCCAC | SEQ ID NO: 16 |
| | GCGAG | |
| glxR R2 | GACTCTAGAGGATCCCCATCGACGGTGTCA | SEQ ID NO: 17 |
| | CCCCAC | |
| glxR(E45A) L2 | GTAAAGGCGGTCACCTGGCgCGCCCTCGTC | SEQ ID NO: 18 |
| | GAAGATGG | |
| glxR(E45A) R1 | CCATCTTCGACGAGGGCGcGCCAGGTGACC | SEQ ID NO: 19 |
| | GCCTTTAC | |
| pDZ_F | TATTACGCCAGCTGGCGAAAG | SEQ ID NO: 20 |
| pDZ_R | TTCCGGCTCGTATGTTGTGTG | SEQ ID NO: 21 |
| glxR(E45S) L2 | GTAAAGGCGGTCACCTGGagaGCCCTCGTC | SEQ ID NO: 22 |
| | GAAGATGG | |
| glxR(E45S) R1 | CCATCTTCGACGAGGGCtctCCAGGTGACCG | SEQ ID NO: 23 |
| | CCTTTAC | |
| glxR(E45F) L2 | GTAAAGGCGGTCACCTGGaaaGCCCTCGTC | SEQ ID NO: 24 |
| | GAAGATGG | |
| glxR(E45F) R1 | CCATCTTCGACGAGGGCtttCCAGGTGACCGC | SEQ ID NO: 25 |
| | CTTTAC | |
| glxR(E45K) L2 | GTAAAGGCGGTCACCTGGCTtGCCCTCGTCG | SEQ ID NO: 26 |
| | AAGATGG | |
| glxR(E45K) R1 | CCATCTTCGACGAGGGCaAGCCAGGTGACC | SEQ ID NO: 27 |
| | GCCTTTAC | |
| glxR(D66A) L2 | GGTTTTCGCGGCCtgCCGGTGCGTGGCGCG | SEQ ID NO: 28 |
| | CAAG | |
| glxR(D66A) R1 | GCGCCACGCACCGGcaGGCCGCGAAAACCT | SEQ ID NO: 29 |
| | GCTG | |
| glxR(D66F) L2 | CAGCAGGTTTTCGCGGCCaaaCGGTGCGTG | SEQ ID NO: 30 |
| | GCGC | |
| glxR(D66F) R1 | GCGCCACGCACCGtttGGCCGCGAAAACCTG | SEQ ID NO: 31 |
| | CTG | |
| glxR(D66K) L2 | GGTTTTCGCGGCCtttCCGGTGCGTGGCGCG | SEQ ID NO: 32 |
| | CAAG | |
| glxR(D66K) R1 | GCGCCACGCACCGaaaGGCCGCGAAAACCT | SEQ ID NO: 33 |
| | GCTG | |
| glxR(T166D) L2 | GCCAGCTTCTTGGtcGCCGAAGCGGTTAGCC | SEQ ID NO: 34 |
| | AGC | |
| glxR(T166D) R1 | AACCGCTTCGGCgaCCAAGAAGCTGGCGCG | SEQ ID NO: 35 |
| | CTG | |
| glxR(T166F) L2 | GCCAGCTTCTTGGaaGCCGAAGCGGTTAGC | SEQ ID NO: 36 |
| | CAGC | |
| glxR(T166F) R1 | AACCGCTTCGGCttCCAAGAAGCTGGCGCGC | SEQ ID NO: 37 |
| | TG | |
| glxR(T166K) L2 | GCCAGCTTCTTGtttGCCGAAGCGGTTAGCCA | SEQ ID NO: 38 |
| | GC | |
| glxR(T166K) R1 | AACCGCTTCGGCaaaCAAGAAGCTGGCGCG | SEQ ID NO: 39 |
| | CTG | |
| glxR(E168A) L2 | AGCGCGCCAGCagCTTGGGCGCCGAAGCGG | SEQ ID NO: 40 |
| | TTAG | |
| glxR(E168A) R1 | CTTCGGCGCCCAAGctGCTGGCGCGCTGCG | SEQ ID NO: 41 |
| | CGTG | |
| glxR(E168F) L2 | AGCGCGCCAGCaaaTTGGGCGCCGAAGCGG | SEQ ID NO: 42 |
| | TTAG | |
| glxR(E168F) R1 | CTTCGGCGCCCAAtttGCTGGCGCGCTGCGC | SEQ ID NO: 43 |
| | GTG | |
| glxR(E168K) L2 | AGCGCGCCAGCcttTTGGGCGCCGAAGCGGT | SEQ ID NO: 44 |
| | TAG | |
| glxR(E168K) R1 | CTTCGGCGCCCAAaagGCTGGCGCGCTGCG | SEQ ID NO: 45 |
| | CGTG | |

A total of 4 kinds of vectors pDZ-gIxR (E45A), pDZ-gIxR (D66A), pDZ-gIxR (T166D) and pDZ-gIxR (E168A) comprising a variant polypeptide in which the 45^{th}, 66^{th}, 166^{th} an 168^{th} amino acids of SEQ ID NO: 1 were substituted with other amino acids were constructed as above, and these vectors are vectors which can substitute the 45^{th} amino acid glutamate (glutamic acid, E) of the GlxR protein into alanine (A), the 66^{th} amino acid aspartate (aspartic acid, D) into alanine (A), the 166^{th} amino acid threonine (T) into aspartate (D), and the 168^{th} amino acid glutamate (E) into alanine (A), respectively.

### Example 2: Evaluation of L-Threonine Productivity of gIxR Variant Strain Based on Wildtype Corynebacterium sp. Microorganism

In the present examples, based on Corynebacterium glutamicum ATCC13032, the L-threonine productivity of the gIxR variant strain in which the 45^{th} amino acid glutamate, the 66^{th} amino acid aspartate, the 166^{th} amino acid threonine, and the 168^{th} amino acid glutamate were substituted with alanine, alanine, aspartate and glutamate, respectively, in the amino acid sequence (SEQ ID NO: 1) of the wildtype GlxR protein endogenously possessed by the ATCC13032 strain, was tried to be evaluated.

The pDZ-gIxR (E45A), pDZ-gIxR (D66A), pDZ-gIxR (T166D) and pDZ-gIxR (E168A) vectors constructed in Example 1 were transformed by electric pulse method. The strains in which a heterogenous nucleotide substitution mutation was introduced into the gIxR gene in this way were named ATCC13032 ΔgIxR::gIxR (E45A), ATCC13032 ΔgIxR::gIxR (D66A), ATCC13032 ΔgIxR::gIxR (T166D), ATCC13032 ΔgIxR::gIxR (E168A), respectively. 4 strains constructed using the ATCC13032 strain as a control group [ATCC13032 ΔgIxR::gIxR (E45A), ATCC13032 ΔgIxR::gIxR (D66A), ATCC13032 ΔgIxR::gIxR (T166D), ATCC13032 ΔgIxR::gIxR (E168A)] were cultured in the following method to measure the rate of sugar consumption, and threonine and lysine production yield.

At first, each strain was inoculated in a 250 [symbol] corner-baffled flask containing seed medium 25 [symbol] , and was cultured with shaking at 200 rpm at 30° C. for 20 hours. Then, [symbol] seed culture solution was inoculated in a 250 [symbol] corner-baffled flask containing production medium 24 [symbol] , and was cultured with shaking at 200 rpm at 32° C. for 24 hours. The composition of the seed medium and production medium was described in Table 4 below, respectively. After completing culturing, the concentration of L-lysine and L-threonine was measured using HPLC (Waters 2478), and the equivalent remaining in the medium (residual equivalent) was analyzed using Biochemistry analyzer (YSI 2900) to measure the rate of sugar consumption, and the result was shown in Table 5.

TABLE 4

| Medium type | Component |
|---|---|
| Seed medium | Glucose 20 g, peptone 10 g, yeast extract 5 g, urea 1.5 g, KH₂PO₄ 4 g, |
| (pH 7.0) | K₂HPO₄ 8 g, MgSO₄•7H₂O 0.5 g, biotin 100 μg, thiamine HCl 1000 μg, |
| | calcium-pantothenic acid 2000 μg, nicotinamide 2000 μg (based on |
| | distilled water 1 liter) |
| L- | Glucose 30 g, KH₂PO₄ 2 g, Urea 3 g, (NH₄)₂SO₄ 40 g, Peptone 2.5 g, |
| THREONINE | CSL(Sigma) 5 g(10 ml), MgSO₄•7H₂O 0.5 g, Leucine 400 mg, CaCO₃ 20 g |
| production | (based on distilled water 1 liter) |
| medium (pH | |
| 7.2) | |

TABLE 5

| | L-Thr | Rate of sugar | L-LYS |
|---|---|---|---|
| | concentration | consumption | concentration |
| Strain | (g/L) | (g/hr) | (g/L) |
| ATCC13032 | 0 | 3.65 | 1.2 |
| ATCC13032 ΔglxR::glxR* (E45A) | 0.22 | 4.22 | 0 |
| ATCC13032 ΔglxR::glxR* (D66A) | 0.16 | 3.98 | 0.2 |
| ATCC13032 ΔglxR::glxR* (T166D) | 0.1 | — | 1 |
| ATCC13032 ΔglxR::glxR* (E168A) | 0.06 | — | 0.7 |

As shown in Table 5, when the 45^{th}, 66^{th}, 166^{th} and 168^{th} amino acids of the GlxR protein were substituted, respectively, compared to the Corynebacterium glutamicum ATC13032 strain, the concentration of the produced L-threonine was increased and the concentration of the by-product L-lysine was reduced.

In addition, as shown in Table 5, it could be confirmed that the rate of sugar consumption was also improved, when the 45^{th} or 66^{th} amino acid of Glx protein was substituted with alanine. Accordingly, as the strain in which the variant in which the 45^{th} or 68^{th} amino acid was substituted with alanine compared to the mother strain (E45A or D66A) had the faster rate of sugar consumption, the time it takes to produce the same amount of L-threonine was shortened, and the L-threonine productivity was improved.

### Example 3: Construction of Vector Library in which the 45, 66, 166 and 168^{th} Amino Acid of gIxR Gene were Substituted with Amino Acids of Different Properties

It was confirmed that the rate of sugar consumption and productivity of L-threonine were improved, when the 45, 66, 166 and 168^{th} amino acids of the GlxR protein were substituted with other amino acids each in Example 2. In order to further confirm the effect when the 45, 66, 166 and 168^{th} amino acids of the GlxR protein (SEQ ID NO: 1) were substituted with amino acids of other properties, substitution of the wildtype amino acids with amino acids comprising the mutation was tried. In order to introduce a total of 13 kinds of heterogenous nucleotide substitution mutations comprising E45A, D66A, T166D and E168A mutations confirmed in Example 2, each recombinant vector was constructed by the same method as Example 1.

pDZ-gIxR (E45S) using primers of SEQ ID NOs: 16 and 22, 17 and 23, pDZ-gIxR (E45F) using primers of SEQ ID NOs: 16 and 24, 17 and 25, pDZ-gIxR (E45K) using primers of SEQ ID NOs: 16 and 26, 17 and 27, pDZ-gIxR (D66F) using primers of SEQ ID NOs: 16 and 30, 17 and 31, pDZ-gIxR (D66K) using primers of SEQ ID NOs: 16 and 32, 17 and 33, pDZ-gIxR (T166F) using primers of SEQ ID NOs: 16 and 36, 17 and 37, pDZ-gIxR (T166K) using primers of SEQ ID NOs: 16 and 38, 17 and 39, pDZ-gIxR (E168F) using primers of SEQ ID NOs: 16 and 42, 17 and 43, and pDZ-gIxR (E168K) using primers of SEQ ID NOs: 16 and 44, 17 and 45 were constructed, respectively. The nucleic acid sequences of the primers used in the present example were described in Table 3 above.

### Example 4: Analysis of L-Threonine Productivity for the 45, 66, 166 and 168th Amino Acid Variants of gIxR Gene Based on Wildtype Corynebacterium sp. Microorganism

The vector library constructed in Example 3 was transformed into the ATC13032 strain by electric pulse method. The strains in which a heterogenous nucleotide substitution mutation was introduced into the gIxR gene in this way were named ATCC13032 ΔgIxR::gIxR* (E45S), ATCC13032 ΔgIxR::gIxR* (E45F), ATCC13032 ΔgIxR::gIxR* (E45K), ATCC13032 ΔgIxR::gIxR* (D66F), ATCC13032 ΔgIxR::gIxR* (D66K), ATCC13032 ΔgIxR::gIxR* (T166F), ATCC13032 ΔgIxR::gIxR* (T166K), ATCC13032 ΔgIxR::gIxR* (E168F), and ATCC13032 ΔgIxR::gIxR* (E168K).

The ATCC13032 strain was used as a control group and cultured by the same method as Example 2 to measure the productivity of L-threonine and L-lysine and rate of sugar consumption, and the result was described in Table 6.

TABLE 6

| | L-Thr | Rate of sugar | L-LYS |
|---|---|---|---|
| | concentration | consumption | concentration |
| Strain | (g/L) | (g/hr) | (g/L) |
| ATCC13032 | 0 | 3.47 | 0.4 |
| ATCC13032 ΔglxR::glxR* (E45A) | 0.2 | 4.02 | 0.4 |
| ATCC13032 ΔglxR::glxR* (E45S) | 0.1 | 3.50 | 0.3 |
| ATCC13032 ΔglxR::glxR* (E45F) | 0.1 | — | 0.4 |
| ATCC13032 ΔglxR::glxR* (E45K) | 0.07 | 3.66 | 0.3 |
| ATCC13032 ΔglxR::glxR* (D66A) | 0.22 | 3.76 | 0.2 |
| ATCC13032 ΔglxR::glxR* (D66F) | 0.11 | — | 0.1 |
| ATCC13032 ΔglxR::glxR* (D66K) | 0.06 | — | 0.4 |
| ATCC13032 ΔglxR::glxR* (T166D) | 0.18 | 3.86 | 0.4 |
| ATCC13032 ΔglxR::glxR* (T166F) | 0.07 | — | 0.3 |
| ATCC13032 ΔglxR::glxR* (T166K) | 0.04 | 3.52 | 0.3 |
| ATCC13032 ΔglxR::glxR* (E168A) | 0.15 | 3.81 | 0.3 |
| ATCC13032 ΔglxR::glxR* (E168F) | 0.1 | — | 0.2 |
| ATCC13032 ΔglxR::glxR* (E168K) | 0.05 | — | 0.1 |

As the experimental result, when the 45^{th} amino acid glutamate in the GlxR protein (SEQ ID NO: 1) was substituted with alanine, serine, phenylalanine or lysine, the L-threonine (Thr) productivity was given to the strain, and the rate of sugar consumption was improved, and the production of by-products such as lysine was reduced. In addition, when the 66^{th} amino acid aspartate in the GlxR protein (SEQ ID NO: 1) was substituted with alanine, phenylalanine or lysine, the L-threonine productivity was given to the strain, and the rate of sugar consumption was improved, and the production of by-products such as lysine was reduced. Furthermore, when the 166^{th} amino acid threonine in the GlxR protein (SEQ ID NO: 1) was substituted with aspartate, phenylalanine or lysine, the L-threonine productivity was given to the strain, and the rate of sugar consumption was improved, and the production of by-products such as lysine was reduced. Moreover, when the 168^{th} amino acid glutamate in the GlxR protein (SEQ ID NO: 1) was substituted with alanine, phenylalanine or lysine, the L-threonine productivity was given to the strain, and the rate of sugar consumption was improved, and the production of by-products such as lysine was reduced.

### Example 5: Analysis of L-Threonine Productivity and Growth Rate for gIxR Variant Strain Based on Threonine Producing Strain

In the present example, in order to clearly examine the threonine concentration and growth rate according to the gIxR mutation introduction, the productivity of L-threonine was tried to be evaluated by introducing 4 kinds of vectors constructed in Example 1 into a L-threonine producing strain. In order to construct a L-threonine producing strain, by changing the 1128^{th} to 1131^{th} nucleotide sequence of the gene encoding wildtype LysC protein from conventional TTG to AAG to solve feedback inhibition of LysC (aspartate kinase) acting as the first important enzyme in the threonine synthesis pathway from wildtype Corynebacterium glutamicum ATCC13032, a strain in which a variant LysC (L377K) mutation, in which the 377^{th} amino acid leucine from the N-terminus of the wildtype LysC protein was substituted with lysine, was introduced was constructed. The amino acid sequence of LysC (L377K) was described in Table 7 below as SEQ ID NO: 46.

The recombinant vector for mutation introduction was constructed by the following method. In order to construct strains in which LysC (L377K) mutation was introduced, primers of SEQ ID NOs: 47 and 48 in which a restriction enzyme SmaI recognition site was inserted into the 5′ fragment and 3′ fragment at the position about 500 bp away, back and forth, respectively, from the 1128 to 1131th position of the lysC gene using genome DNA extracted from the ATCC13032 strain were synthesized. To introduce LysC (L377K) heterogenous nucleotide substitution mutation, primers of SEQ ID NOs: 49 and 50 to substitute the 1128 to 1131th nucleotide sequence of the lysC gene were synthesized.

Specifically, the pDZ-lysC (L377K) plasmid was constructed in a form in which DNA fragments (515, 538 bp each) positioned at the 5′ and 3′ terminuses of the lysC gene were connected to the pDZ vector. The 5′ terminal gene fragment was constructed using primers of SEQ ID NOs: 47 and 49 using the chromosome of the ATCC13032 strain as a template through PCR. PCR conditions were repeating denaturation at 94° C. for 5 minutes, denaturation at 94° C. for 1 minute, annealing at 56° C. for 1 minute, and polymerization at 72° C. for 40 seconds, 30 times, and then performing polymerization at 72° C. for 10 minutes. By the same method, the gene fragment positioned at the 3′ terminus of the lysC gene was constructed using SEQ ID NOs: 48 and 50 through PCR. After purifying the amplified DNA fragment using Quiagen's PCR Purification kit, it was used as an insertion DNA fragment for vector construction.

On the other hand, the pDZ vector treated with the restriction enzyme SmaI and then heat-treated at 65° C. for 20 minutes and the insertion DNA fragment amplified through PCR were connected using Infusion Cloning Kit, and then transformed into E. coli DH5α. The strain was plated on LB solid medium containing kanamycin (25 mg/l). After selecting colonies transformed with the vector into which the desired gene was inserted through PCR using primers of SEQ ID NOs: 47 and 48, a plasmid was obtained using a commonly known plasmid extraction method. The plasmid was named pDZ-lysC (L377K). The nucleic acid sequence of the primers used in the present example was described in Table 7 below.

TABLE 7

| Name | Sequence | SEQ ID NO |
|---|---|---|
| LysC_L377K | MALVVQKYGGSSLESAERIRNVAERIVATKKAGND | SEQ ID NO: 46 |
| | VVVVCSAMGDTTDELLELAAAVNPVPPAREMDML | |
| | LTAGERISNALVAMAIESLGAEAQSFTGSQAGVLT | |
| | TERHGNARIVDVTPGRVREALDEGKICIVAGFQGV | |
| | NKETRDVTTLGRGGSDTTAVALAAALNADVCEIYS | |
| | DVDGVYTADPRIVPNAQKLEKLSFEEMLELAAVGS | |
| | KILVLRSVEYARAFNVPLRVRSSYSNDPGTLIAGS | |
| | MEDIPVEEAVLTGVATDKSEAKVTVLGISDKPGEA | |
| | AKVFRALADAEINIDMVLQNVSSVEDGTTDITFTCP | |
| | RSDGRRAMEILKKLQVQGNWTNVLYDDQVGKVSL | |
| | VGAGMKSHPGVTAEFMEALRDVNVNIEKISTSEIRI | |
| | SVLIREDDLDAAARALHEQFQLGGEDEAVVYAGT | |
| | GR | |
| LysC L1 | TCGAGCTCGGTACCCGCTGCGCAGTGTTGAATA | SEQ ID NO: 47 |
| | C | |
| LysC R2 | CTCTAGAGGATCCCCGTTCACCTCAGAGACGAT | SEQ ID NO: 48 |
| | T | |
| lysC(L377K) L2 | TGGAAATCTTTTCGATGTTCACGTTGACAT | SEQ ID NO: 49 |
| lysC(L377K) R1 | ATGTCAACGTGAACATCGAAAAGATTTCC | SEQ ID NO: 50 |

The pDZ-lysC (L377K) constructed above was transformed into the Corynebacterium glutamicum ATCC13032 strain by electric pulse method. The strain in which a heterogenous nucleotide substitution mutation was introduced into the lysC gene in this way was named CJP1. The CJP1 was named CA01-2307, and it was deposited to Korean Culture Center of Microorganisms (KCCM) which is an international depository authority under the Budapest Treaty, and it was given an accession number KCCM12000P.

To the KCCM12000P strain constructed above, mutation was introduced into a gene encoding homoserine dehydrogenase (Hom) producing homoserine which is a common intermediate of the biosynthesis pathway of L-threonine and L-isoleucine. Specifically, by substituting the 1218 to 1221th nucleotide sequence of the gene encoding wildtype homoserine dehydrogenase from TTG to AAG, the strain in which variant Hom (R407H) mutation in a form in which the 407^{th} amino acid from the N-terminus of wildtype homoserine dehydrogenase, arginine was substituted with histidine was constructed. The amino acid sequence of Hom (R407H) was described in Table 8 below as SEQ ID NO: 51. The recombinant vector for mutation introduction was constructed by the following method.

At first, primers of SEQ ID NOs: 52 and 53 in which a restriction enzyme SaII recognition site was inserted to the 5′ fragment and 3′ fragment at the position about 600 bp away, back and forth, respectively, from the 1219 to 1221th position of the hom gene using genome DNA extracted from the ATCC13032 strain as a template were synthesized. To introduce Hom (R407H) heterogenous nucleotide substitution mutation, primers of SEQ ID NOs: 54 and 55 to substitute the 1219 to 1221th nucleotide sequence of the hom gene were synthesized.

Specifically, the pDZ-hom (R407H) plasmid was constructed in a form in which DNA fragments (600 bp each) positioned at the 5′ and 3′ terminuses of the hom gene. The 5′ terminal gene fragment was constructed using primers of SEQ ID NOs: 52 and 54 using chromosome of the WT strain through PCR. PCR conditions were repeating denaturation at 94° C. for 2 minutes, denaturation at 94° C. for 1 minute, annealing at 56° C. for 1 minute, and polymerization at 72° C. for 40 seconds, 30 times, and then performing polymerization at 72° C. for 10 minutes. By the same method, the gene fragment positioned at the 3′ terminus of the hom gene was constructed using SEQ ID NOs: 53 and 55 through PCR. After purifying the amplified DNA fragment using Quiagen's PCR Purification kit, it was used as an insertion DNA fragment for vector construction.

On the other hand, the pDZ vector treated with the restriction enzyme SaII and then heat-treated at 65° C. for 20 minutes and the insertion DNA fragment amplified through PCR were connected using Infusion Cloning Kit, and then transformed into E. coli DH5α. The strain was plated on LB solid medium containing kanamycin (25 mg/l). After selecting colonies transformed with the vector into which the desired gene was inserted through PCR using primers of SEQ ID NOs: 52 and 53, a plasmid was obtained using a commonly known plasmid extraction method. The plasmid was named pDZ-hom (R407H). The nucleic acid sequence of the primers used in the present example was described in Table 8 below.

TABLE 8

| Name | Sequence | SEQ ID NO |
|---|---|---|
| Hom_R407H | MTSASAPSFNPGKGPGSAVGIALLGFGTVGTEV | SEQ ID NO: 51 |
| | MRLMTEYGDELAHRIGGPLEVRGIAVSDISKPRE | |
| | GVAPELLTEDAFALIEREDVDIVVEVIGGIEYPREV | |
| | VLAALKAGKSVVTANKALVAAHSAELADAAEAAN | |
| | VDLYFEAAVAGAIPVVGPLRRSLAGDQIQSVMGIV | |
| | NGTTNFILDAMDSTGADYADSLAEATRLGYAEAD | |
| | PTADVEGHDAASKAAILASIAFHTRVTADDVYCE | |
| | GISNISAADIEAAQQAGHTIKLLAICEKFTNKEGKS | |
| | AISARVHPTLLPVSHPLASVNKSFNAIFVEAEAAG | |
| | RLMFYGNGAGGAPTASAVLGDVVGAARNKVHG | |
| | GRAPGESTYANLPIADFGETTTRYHLDMDVEDRV | |
| | GVLAELASLFSEQGISLRTIRQEERDDDAHLIVVT | |
| | HSALESDLSRTVELLKAKPVVKAINSVIRLERD | |
| hom L1 | ATCCTCTAGAGTCGACCCAACTGCAGACGTCG | SEQ ID NO: 52 |
| | AA | |
| hom R2 | ATGCCTGCAGGTCGACATAGACAGATTTGTCC | SEQ ID NO: 53 |
| | ACG | |
| hom(R407H) L2 | GTGACCACGATCAGATGTGCATCATCATCGCG | SEQ ID NO: 54 |
| | CTC | |
| hom(R407H) R1 | GCGATGATGATGCACATCTGATCGTGGTCACC | SEQ ID NO: 55 |
| | CAC | |

The pDZ-hom (R407H) constructed above was transformed into KCCM12000P by electric pulse method. The strain in which a heterogenous nucleotide substitution mutation was introduced into the hom gene in this way was named KCCM12000P-R398Q, and it was deposited to Korean Culture Center of Microorganisms (KCCM) which is an international depository authority under the Budapest Treaty, and it was given an accession number KCCM12120P (Korean Patent No. 10-1947959).

The pDZ-gIxR (E45A), pDZ-gIxR (D66A), pDZ-gIxR (T166D) and pDZ-gIxR (E168A) vectors constructed in Example 1 were transformed to the KCCM12120P strain by electric pulse method, respectively. The strain in which nucleotide substitution mutation was introduced to the gIxR gene was constructed, and KCCM12120P ΔgIxR::gIxR* (E45A) was named CA09-2373, and KCCM12120P ΔgIxR::gIxR* (D66A) was named CA09-2374, and KCCM12120P ΔgIxR::gIxR* (T166D) was named CA09-2325, and KCCM12120P ΔgIxR::gIxR* (E168A) was named CA09-2324, respectively.

A total of 4 kinds of gIxR variants were cultured by the following method using a L-threonine producing strain, KCCM12120P strain, to measure the L-threonine production yield, rate of sugar consumption and production concentration of by-products such as L-lysine.

Each strain was inoculated in a 250 [symbol] corner-baffled flask containing seed medium 25 [symbol] , and was cultured with shaking at 200 rpm at 30° C. for 20 hours. Then, [symbol] seed culture solution was inoculated in a 250 [symbol] corner-baffled flask containing production medium 24 [symbol] , and was cultured with shaking at 200 rpm at 32° C. for 48 hours. The composition of the seed medium and production medium was described in Table 9 below, respectively. After completing culturing, the concentration of L-lysine and L-threonine and rate of sugar consumption were measured using HPLC and Biochemistry analyzer (YSI 2900), and the result was shown in Table 10.

TABLE 9

| Medium type | Component |
|---|---|
| Seed medium (pH | Glucose 20 g, peptone 10 g, yeast extract 5 g, urea 1.5 g, KH₂PO₄ 4 |
| 7.0) | g, K₂HPO₄ 8 g, MgSO₄•7H₂O 0.5 g, biotin 100 μg, thiamine HCl 1000 |
| | μg, calcium-pantothenic acid 2000 μg, nicotinamide 2000 μg (based |
| | on distilled water 1 liter) |
| L-THREONINE | Glucose 100 g, KH₂PO₄ 2 g, Urea 3 g, (NH₄)₂SO₄ 25 g, Peptone 2.5 g, |
| production medium | CSL(Sigma) 5 g(10 ml), MgSO₄•7H₂O 0.5 g, biotin 100 μg, thiamine |
| (pH 7.0) | HCl 1000 μg, calcium-pantothenic acid 2000 μg, nicotinamide 3000 |
| | μg, CaCO₃ 30 g (based on distilled water 1 liter) |

TABLE 10

| | | Rate of | |
|---|---|---|---|
| | L-Thr | sugar | L-LYS |
| | concentration | consumption | concentration |
| Strain | (g/L) | (g/hr) | (g/L) |
| KCCM12120P | 7.62 | 4.04 | 0.36 |
| CA09-2373 | 9.23 | 4.66 | 0.28 |
| CA09-2374 | 9.67 | 4.21 | 0.31 |
| CA09-2325 | 8.87 | 4.13 | 0.35 |
| CA09-2324 | 8.90 | 4.22 | — |

As shown in Table 10, as the result of measuring the production concentration of L-threonine and by-products (L-lysine) by culturing 4 kinds of gIxR variant strains (CA09-2373, CA09-2374, CA09-2325, CA09-2324) using the L-threonine producing strain, KCCM12120P strain as a control group, it could be confirmed that all 4 kinds of strains in which the substitution mutation was introduced to the gIxR gene reduced the production of other by-products including L-lysine or there was no big change compared to the control group, but the production of L-threonine was significantly increased. In addition, it could be confirmed that in all CA09-2373, CA09-2374, CA09-2325, and CA09-2324 strains, compared to the control group, the rate of sugar consumption was improved, and the time it took to produce the same amount of L-threonine was shortened and the L-threonine productivity was improved.

The CA09-2373, CA09-2374, CA09-2325 and CA09-2324 strains were deposited to Korean Culture Center of Microorganisms (KCCM) which is an international depository authority under the Budapest Treaty, and they were given accession numbers KCCM12901P, KCCM12902P, KCCM12900P, and KCCM12899P, respectively.

### Accession Number

Depository authority name: Korean Culture Center of Microorganisms (overseas)
Accession number: KCCM12899P
Accession date: 20201217
Depository authority name: Korean Culture Center of Microorganisms (overseas)
Accession number: KCCM12900P
Accession date: 20201217
Depository authority name: Korean Culture Center of Microorganisms (overseas)
Accession number: KCCM12901P
Accession date: 20201217
Depository authority name: Korean Culture Center of Microorganisms (overseas)
Accession number: KCCM12902P
Accession date: 20201217

1. A GlxR (glyoxylate bypass regulator) protein variant, comprising a mutation selected from the group consisting of (1) to (4) below:
(1) substitution of the residue corresponding to the 45^{th} amino acid position from the N-terminus in the amino acid sequence of SEQ ID NO: 1 with alanine, serine, phenylalanine or lysine;
(2) substitution of the residue corresponding to the 66^{th} amino acid position from the N-terminus in the amino acid sequence of SEQ ID NO: 1 with alanine, phenylalanine or lysine;
(3) substitution of the residue corresponding to the 166^{th} amino acid position from the N-terminus in the amino acid sequence of SEQ ID NO: 1 with aspartic acid, phenylalanine or lysine; and
(4) substitution of the residue corresponding to the 168^{th} amino acid position from the N-terminus in the amino acid sequence of SEQ ID NO: 1 with alanine, phenylalanine or lysine.

2. A polynucleotide, encoding the GlxR protein variant of claim 1.

3. A microorganism, comprising at least one selected from the group consisting of the GlxR protein variant of claim 1, and a polynucleotide encoding the GlxR protein variant.

4. The microorganism according to claim 3, wherein the microorganism has L-threonine productivity.

5. The microorganism according to claim 3, wherein the microorganism is Corynebacterium sp.

6. The microorganism according to claim 5, wherein the Corynebacterium sp. microorganism is Corynebacterium glutamicum.

7. A method for producing L-threonine, comprising a step of culturing the microorganism of claim 3 in a medium.

8. The method according to claim 7, wherein the method further comprises a step of recovering L-threonine form the cultured medium or microorganism.

9. The method according to claim 7, wherein the microorganism is Corynebacterium sp.

10. The method according to claim 9, wherein the Corynebacterium sp. microorganism is Corynebacterium glutamicum.
